(19) United States
(12) Patent Application Publication
(10) Pub. No.: US 2025/0221963 A1
(43) Pub. Date: Jul. 10, 2025

(54) 5-MEO-DMT FOR USE IN THE TREATMENT OF POSTPARTUM DEPRESSION

(71) Applicant: GH RESEARCH IRELAND LIMITED, Dublin 2 (IE)

(72) Inventors: Theis TERWEY, Berlin (DE); Conor BURKE, Laytown (IE); Naoise GAFFNEY, Dublin (IE)

(73) Assignee: GH RESEARCH IRELAND LIMITED, Dublin 2 (IE)

(21) Appl. No.: 18/851,356

(22) PCT Filed: Mar. 27, 2023
(86) PCT No.: PCT/EP2023/057873

§ 371 Date: Sep. 26, 2024

(30) Foreign Application Priority Data
Mar. 27, 2022 (EP) 22000083.0
Mar. 27, 2022 (EP) 22000086.3
Jan. 30, 2023 (EP) 23153939.6
Jan. 30, 2023 (EP) 23153995.8

(51) Int. Cl. A61K 31/4045; A61P 25/24

(52) U.S. Cl. CPC A61K 31/4045; A61P 25/24

(57) ABSTRACT

5-Methoxy-N,N-dimethyltryptamine (5-MeO-DMT) or a pharmaceutically acceptable salt thereof is used in treating a patient suffering from postpartum depression (PPD).

### TECHNICAL FIELD

The present invention is directed to improved methods for the treatment of postpartum depression (PPD) comprising administering to a patient in need thereof a therapeutically effective amount of 5-methoxy-N,N-dimethyltryptamine (5-MeO-DMT) or of a pharmaceutically acceptable salt thereof. The invention also allows for treating PPD in a breast-feeding mother without substantial interruption of breastfeeding.

### BACKGROUND OF THE INVENTION

Over 50% of women may experience short-lasting low mood or tearfulness after childbirth. However, a subset of women may develop PPD—a debilitating mood disorder occurring during pregnancy or within 4 weeks following delivery.

Epidemiological studies estimate that the prevalence rate of PPD is about 15%.

Research has demonstrated that PPD leads to a wide range of negative consequences for the affected mother, her infant(s) and her family. For example, women with PPD may develop thoughts of self-harm or harming their child and they are at increased risk of suicide. PPD may further lead to disruptions in the interactions between mother and child, exemplified by higher rates of disengaged behaviour and lower rates of visual and vocal communication between mother and child. Evidence also suggests an association between PPD and child development, as illustrated by the fact that children of patients suffering from PPD have a greater risk of impaired cognitive development.

Over 20% of women diagnosed with PPD remain depressed after 12 months of follow-up and 13% continue to suffer from PPD 2 years after being diagnosed. Furthermore, evidence suggests that 40% of women relapse and that untreated cases of PPD may lead to recurring bouts of depression.

Thus, it is evident that the burden of PPD is significant from multiple perspectives and that successful detection and treatment of PPD is of vital importance.

Despite this evident need, treatment options are rather limited. Quite generally, known treatments for depression are associated with only limited treatment success, in particular in patients suffering not merely from mild symptoms of the disease. In case of PPD, a compounding factor is that patients will often be breastfeeding. For many medicaments, lactating women are advised to discontinue breastfeeding during the period in which they take the medicament and for some time thereafter as the medicament may be excreted in milk and expose the suckling child to a risk.

Moreover, research has shown that breastfeeding mothers may be reluctant to commence pharmacological treatment due to a range of concerns.

In consequence, breastfeeding PPD patients may be confronted with a situation where a decision must be made whether to discontinue breast-feeding or to discontinue/abstain from therapy.

Currently, PPD is primarily treated via psychological therapies or pharmacotherapy. National Institute for Health and Care Excellence (NICE) guidelines recommend that initiation of treatment be preceded by discussions with the patient regarding the higher threshold for pharmacological interventions. Such treatments may consist of antidepressants such as selective serotonin reuptake inhibitors (SSRIs), some of which are not contraindicated during breastfeeding.

More recently in the U.S., brexanolone (Zulresso) received FDA approval—thus making it the first pharmacological therapy indicated specifically for PPD. Brexanolone is a positive allosteric modulator of GABAa receptors which is administered via a 60-hour infusion. The efficacy of brexanolone was displayed in two phase 3 trials—one of which showed significantly reduced Hamilton Rating Scale for Depression (HAM-D) scores 30 days after initiation of the infusion and one of which failed to show efficacy beyond 7 days. However, brexanolone requires a hospital admission for the 60-hour infusion, and significant side effects occur.

A further noteworthy fact is that patients were required to cease breastfeeding during the brexanolone infusions and, in the aftermath of its approval, some insurance companies are requiring that this criterion be upheld in patients planned for treatment.

Against this background there is a need for an improved treatment of PPD, in particular a treatment that not only effectively addresses depression and leads to a rapid clinical response but also avoids interference with the patient's everyday activities, in particular regarding care of the infant(s). The treatment should improve maternal functioning. There is furthermore a need for a treatment of PPD that does not require a substantial interruption of breastfeeding.

While there has recently been significant interest in hallucinogens for the treatment of mental disorders, this has so far not led to a treatment of PPD. This is due to a general lack of relevant clinical data which would allow drawing conclusions on the clinical utility of hallucinogens in PPD as well as due to specific concerns that administration of hallucinogens may not be appropriate for breastfeeding mothers.

Hallucinogens including psychedelics are chemical compounds, some naturally occurring, some synthetic, which are defined by their ability to induce in humans after consumption sensory distortions, such as changes in auditory and visual perception, as well as distortions of mood and cognition. The term hallucinogen encompasses a rather broad group of psychoactive molecules with different modes of action. Some mental disorders have been suggested as in principle being amenable for treatment with psychoactive molecules, like psychedelics.

However, no psychedelic drug has been approved by any regulatory agency. In fact, clinical experience with such molecules is still rather restricted.

One compound already investigated in clinical trials is 5-methoxy-N,N-dimethyltryptamine (5-MeO-DMT). WO 2020/169850 reports on tests in healthy volunteers as well as a clinical trial involving patients suffering from treatment resistant depression (TRD), i.e., a form of major depressive disorder. Patients suffering from PPD were not included in the trial.

Against this background, an aim of the invention is in particular the provision of therapies which are more effective (i.e., a) larger percentage of patients experiencing a clinical response, b) a larger average clinical response, c) an earlier onset of the clinical response, and/or d) a more durable clinical response) than previously described therapies.

A further aim of the current invention is to provide a compound for improved psychoactive therapies and dosing regimens for said therapies which have a better safety profile and/or are better tolerated than previously described therapies. Another aim of the current invention is to provide a compound for improved psychoactive therapies and dosing regimens for said therapies which are more convenient than previously described therapies. Another aim of the current invention is to provide a compound for improved psychoactive therapies and dosing regimens for said therapies which are associated with higher rates of patient compliance (including higher rates of treatment initiation) than previously described therapies. A still further aim of the current invention is to identify specific disease aspects and specific subgroups of disease aspects which benefit from such improved psychoactive therapies.

Still further aims of the invention are to improve maternal functioning in patients suffering from PPD. Improvement of maternal functioning in a breastfeeding mother diagnosed with a mental disorder is also an aim of the invention.

It is moreover an aim of the invention to provide a treatment for breastfeeding mothers diagnosed with PPD or with another mental disorder which allows them to continue breastfeeding without significant interruption due to the treatment.

### SUMMARY OF THE INVENTION

The present invention relates to 5-methoxy-N,N-dimethyltryptamine (5-MeO-DMT) or a pharmaceutically acceptable salt thereof for use in treating postpartum depression (PPD). The treatment not only improves depressive symptoms but also maternal functioning.

The invention also allows for treating PPD in a breastfeeding mother without substantial interruption of breastfeeding.

### DETAILED DESCRIPTION OF THE INVENTION

### Definitions

As used in the context of the present invention, unless otherwise noted, the term “5-MeO-DMT” refers to the free base 5-MeO-DMT. It is contemplated that pharmaceutically acceptable salts of 5-MeO-DMT may also be used. Such salts are in particular acid addition salts, wherein the acid may be selected from, for instance, acetic acid, benzoic acid, citric acid, fumaric acid, hydrobromic acid, hydrochloric acid, hydrofluoric acid, hydroiodic acid, oxalic acid, succinic acid and triflic acid. A preferred example is the hydrobromide salt. The appropriate weight amount of a salt to be administered can be calculated from the weight amount of the free base, assuming that equimolar amounts are used.

As used in the context of the present invention, a “patient” to be treated is a woman who is diagnosed with postpartum depression (PPD) according to established medical standards. The diagnosis will be by a physician or a psychologist. It is not sufficient that the human subject herself considers that she is suffering from the disorder.

The patient may suffer from treatment resistant disease. As used herein, “treatment resistance” means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy.

As used in the context of the present invention, “suicidal ideation” refers to thinking about, considering, or planning for suicide. The presence of suicidal ideation in a patient will be diagnosed by a physician or a psychologist, using established protocols and methods for diagnosing suicidality. It is generally not sufficient that the patient himself considers that he is suffering from suicidal ideation. In some situations, a patient experiencing suicidal ideation will be at imminent risk of committing suicide, or will be considered to have ‘intent to act.’

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder.

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the disease, condition or disorder being treated.

“Clinical response” includes, but is not limited to, improvements on rating scales such as the Clinical Global Impression-Severity scale (CGI-S), the Patient Global Impression-Severity scale (PGI-S), the Clinical Global Impression-Improvement scale (CGI-I) or the Patient Global Impression-Improvement scale (PGI-I) and further includes, but is not limited to, endpoints such as the Montgomery-Åsberg Depression Rating Scale (MADRS), the 17-item Hamilton Depression Rating Scale (HAM-D) or the Edinburgh Postnatal Depression Scale (EPDS). Further relevant scales to assess clinical outcome include the Clinician Administered Dissociative States Scale (CADSS), the Brief Psychiatric Rating Scale (BPRS) and the Columbia-Suicide Severity Rating Scale (C-SSRS).

Maternal functioning may be assessed using the Barkin Index of Maternal Functioning (BIMF).

Individual items of the scales indicated as well as sub-combinations of individual items may be used to assess specific disease aspects.

When assessing a clinical response at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied.

The considerations outlined apply for early timepoints because, on the one hand, in order to assess a clinical response, the influence of the patient's status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if necessary, an adapted recall period, so that it is not necessary to carry forward any pre-treatment score. For instance, if the BIMF is assessed on day 7, a recall period of seven days will be used (instead of the standard recall period of 2 weeks).

As used in the context of the present invention, unless otherwise noted, the term “administration” (or “application”) shall mean the introduction of an amount, which may be a predetermined amount, of active compound or pharmaceutical ingredient into a patient via any route. Preferably, the active compound is administered by inhalation, nasally, by buccal administration or by sublingual administration.

As used in the context of the present invention, unless otherwise noted, the terms “dose” and “dosage” and “dosage amount” shall mean the amount of active compound or pharmaceutical ingredient which is administered to a patient in an individual administration. The term “dosage regimen” (or “dosing regimen”) shall mean a defined sequence of one or more individual administrations.

As used herein, “aerosol” means a stable system consisting of a gaseous medium (a pharmaceutically acceptable gas, such as air) and miniscule suspended solid and/or liquid particles. The term “degradation product” refers to a compound resulting from a chemical modification of 5-MeO-DMT as a result of a chemical reaction during aerosol formation. Such reaction includes, without limitation, oxidation. When a percentage of a “degradation product” is described in the context of the present invention, then this refers to the quantity of 5-MeO-DMT degradation products present in a sample divided by the quantity of 5-MeO-DMT plus 5-MeO-DMT degradation products present in the sample multiplied by 100%, i.e., (Sum of quantities of all 5-MeO-DMT degradation products present in the sample)/((Quantity of 5-MeO-DMT present in the sample)+(Sum of quantities of all 5-MeO-DMT degradation products present in the sample))×100%. As used herein, the term “impurity” refers to unwanted compounds contaminating a sample of 5-MeO-DMT (or of a pharmaceutically acceptable salt thereof). Impurities may be contained in the starting material before aerosol formation or may be degradation products.

The term “purity” refers to 100% minus the percent of all 5-MeO-DMT degradation products and all other impurities present, i.e., 100%−(Sum of quantities of all 5-MeO-DMT degradation products present+Sum of quantities of all other impurities present)/(Quantity of 5-MeO-DMT present+Sum of quantities of all 5-MeO-DMT degradation products present+Sum of quantities of all other impurities present)×100%.

The term “mass median aerodynamic diameter” (MMAD), is the diameter at which 50% of the particles present in an aerosol are larger than this calculated diameter, and 50% are smaller. The term “aerosol particle mass density” refers to the mass of aerosol particles per unit volume of aerosol. The term “aerosol particle formation rate” refers to the aerosolized mass of 5-MeO-DMT per unit of aerosolization time.

### Postpartum Depression

Postpartum depression (PPD) is a complex mix of physical, emotional, and behavioral changes that happen in some women after giving birth. PPD is also known as major depressive disorder with peripartum onset. According to DSM-5 (Diagnostic and Statistical Manual of Mental Disorders, 5th Edition) criteria, PPD is diagnosed when major depressive disorder (MDD) symptoms begin during pregnancy or within four weeks of delivery.

A patient treated according to the present invention is preferably a woman diagnosed with PPD and >4 weeks postpartum. Further, the patient will preferably be •9 months postpartum.

Depressive aspects of PPD may be assessed by the HAM-D or the MADRS score. The Edinburgh Postnatal Depression Scale (EPDS) can also be used.

The Montgomery-Åsberg Depression Rating Scale (MADRS) is a ten-item diagnostic questionnaire used to measure the severity of depressive episodes in patients with mood disorders (Montgomery, S. A., & Åsberg, M. (1979). A new depression scale designed to be sensitive to change. The British Journal of Psychiatry 134, p. 382). It was designed as an adjunct to the Hamilton Rating Scale for Depression (HAM-D), which would be more sensitive to the changes brought on by antidepressants and other forms of treatment. Higher MADRS score indicates more severe depression. The items considered are apparent sadness; reported sadness; inner tension; reduced sleep; reduced appetite; concentration difficulties; lassitude; inability to feel; pessimistic thoughts; and suicidal thoughts, and each item yields a score of 0 to 6. The overall score ranges from 0 to 60.

A patient may suffer from moderate or severe PPD as indicated by a Montgomery-Åsberg Depression Rating Scale (MADRS) score of 20 or more or by a Hamilton Depression Rating Scale (HAM-D) score of 16 or more. It is further considered that the patient may suffer from severe PPD as indicated by a Montgomery-Åsberg Depression Rating Scale (MADRS) score of 35 or more or by a Hamilton Depression Rating Scale (HAM-D) score of 27 or more. The patient may be diagnosed with a treatment-resistant form of PPD.

A patient treated according to the invention may have a Montgomery-Åsberg Depression Rating Scale (MADRS) score of 20 or more or a 17-item Hamilton Depression Rating Scale (HAM-D) score of 16 or more.

Further, a patient treated according to the invention may have a MADRS score of 28 or more or a HAM-D score of 22 or more.

Still further, a patient treated according to the invention may have a MADRS score of 35 or more or a HAM-D score of 25 or more.

In addition to the above, the inventors consider that PPD compromises maternal functioning. In particular the first year after childbirth marks a critical window for both mother and child. In most cases, mothers are the primary caregivers and are, therefore, responsible for the majority of the work related to infant care tasks.

Maternal functioning includes aspects of maternal competence relating to interactions with the infant(s) as well as maternal self-care.

Maternal functioning, including the emotional aspect of mothering, is also important for the child's development. In fact, the quality of mother-child interaction in the year after birth affects infant development. High levels of maternal functioning are likely to correlate with positive infant development outcomes. Likewise, impaired functioning in the postpartum period might impede optimal infant development.

The Barkin Index of Maternal Functioning (BIMF) was designed to measure functioning in the year after childbirth. The BIMF is a 20-item self-report measure of functioning. Each item is assigned a score between 0 and 6 so that the maximum total score is 120. The higher the score, the better maternal functioning is rated.

The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, score of 80 or below is considered herein as representing compromised maternal functioning, a score of 65 or below is considered herein as representing severely compromised maternal functioning. The invention in particular allows improving maternal functioning in patients having a score of 80 or below before treatment and in patients having a score of even 65 or below.

### The Active Agent

The above discussion shows that PPD is characterized by several aspects which as such present a significant disease burden and deserve appropriate treatment. Thus, there is not only a need for a treatment, in particular by pharmacological intervention, to improve overall disease scores but also to improve specific aspects of the disease.

The inventors considered that a carefully chosen hallucinogen may lead to an improved treatment of important aspects of PPD and may lead to overall improvements of the disease and of maternal functioning.

The inventors further considered that a carefully chosen hallucinogen may allow continuing breastfeeding without substantial interruption in case of treatment of a breastfeeding mother suffering from PPD.

One group of hallucinogens entails compounds which bind to the 5-hydroxytryptamine (5-HT) receptors, which are also referred to as serotonin receptors (described are 7 families 5-HT1 to 5-HT7 with several subtypes). Examples are lysergic acid diethylamide (LSD), psilocybin, and N,N-dimethyltryptamine (DMT). These serotonergic agents are often referred to as “psychedelics”, which emphasizes their predominant ability to induce qualitatively altered states of consciousness such as euphoria, trance, transcendence of time and space, spiritual experiences, dissolution of self-boundaries, or even near-death experiences, while other effects such as sedation, narcosis, or excessive stimulation are only minimal.

Chemically, serotonergic psychedelics are either phenylalkylamines or indoleamines, with the indoleamine class being divided into two subsets, ergolines and tryptamines, the latter being derived from tryptamine.

The various serotonergic psychedelics have different binding affinity and activation potency for various serotonin receptors, particularly 5-HT1A, 5-HT2A, and 5-HT2C, and their activity may also be modulated by interaction with other targets such as monoamine transporters and trace amine-associated receptors.

Recently published clinical studies which have used serotonergic psychedelic drugs such as LSD, psilocybin and DMT (using the shamanic brew Ayahuasca, which contains DMT) in certain mental disorders suggest that those compounds could provide an alternative to the currently available treatments for certain mental disorders. However, there are reports that these compounds can induce mania in patients suffering from depressive symptoms, and this may preclude their clinical use.

For instance, Lake et al. (Lake, C. R., Stirba, A. L., Kinneman, R. E. Jr, Carlson, B., Holloway, H. C., 1981. Mania associated with LSD ingestion. American Journal of Psychiatry. 138(11):1508-9) report about a patient who suffered a manic attack after ingesting LSD or an LSD analogue. The patient experienced acute symptoms of LSD intoxication, which resolved but were followed in about 3 weeks by a typical manic episode of psychotic magnitude. Hendin and Penn (Hendin, H. M., Penn, A. D., 2021. An episode of mania following self-reported ingestion of psilocybin mushrooms in a woman previously not diagnosed with bipolar disorder: A case report. Bipolar Disorders 23(4):1-3) report about an episode of mania following self-reported ingestion of psilocybin mushrooms. Szmulewicz et al. (Szmulewicz, A. G., Valerio, M. P., and Jose M Smith, J. M., 2015. Switch to mania after ayahuasca consumption in a man with bipolar disorder: a case report. International Journal of Bipolar Disorders (2015) 3:4) report on a switch to mania after consumption of ayahuasca, a DMT containing brew, in a man with bipolar disorder.

A further case report is found in Brown, T., Shao, W., Ayub, S., Chong, D., & Cornelius, C. (2017). A Physician's attempt to self-medicate bipolar depression with N, N-dimethyl-tryptamine (DMT). Journal of Psychoactive Drugs, 49(4), 294-296.

The inventors considered that in order to avoid the induction of mania or hypomania or at least reduce the risk of induction of mania or hypomania, the compound administered must be appropriately chosen and preferably is administered in a particular dosing regimen.

The inventors identified 5-methoxy-N,N-dimethyltryptamine (5-MeO-DMT) as a psychedelic of particular interest for use in therapy. 5-MeO-DMT has a distinct pharmacological profile which differs from that of other psychedelic compounds.

5-MeO-DMT is a potent, fast-acting, naturally occurring serotonin (5-HT) agonist, acting at both the 5-HT1A and the 5-HT2A receptor, with higher affinity for the 5-HT1A receptor subtype compared to other classical psychedelics.

Inhibition constants (Kᵢ values) as further detailed on the example section below for psilocin (the dephosphorylated from of psilocybin which is formed after uptake of psilocybin), DMT and 5-MeO-DMT are 48, 38 and 1.80 nM, respectively, at 5-HT1A receptors located in the hippocampus of post-mortem human brain. Thus, 5-MeO-DMT exhibits high affinity and psilocin and DMT exhibit moderate affinity for 5-HT1A receptors. Inhibition constants (Kᵢ values) for psilocin, DMT and 5-MeO-DMT are 37, 117 and 122 nM, respectively, at 5-HT2A receptors located in the frontal cortex of post-mortem human brain. Therefore, psilocin exhibits moderate/strong affinity and DMT and 5-MeO-DMT exhibit comparatively weak affinity for 5-HT2A receptors.

Relative to the other psychoactive compounds mentioned previously, 5-MeO-DMT displays an enhanced affinity for the 5-HT1A receptor, where it acts as a potent agonist. In the case of psilocin and DMT, there is an increased contribution of 5-HT2A binding, relative to 5-MeO-DMT, with the latter displaying the largest differential affinity for 5-HT1A over 5-HT2A of the three compounds. Therefore, 5-HT1A binding plays a much bigger role in the overall effect of 5-MeO-DMT relative to 5-HT2A binding compared to the other two compounds.

It has been reported that 5-HT1A agonism reduces impulsivity and aggression, whereas 5-HT2A agonism can result in short-term increases in these same traits. Furthermore, the dopamine system has been implicated in contributing to mania, with increased dopamine drive being linked to mania. LSD, psilocybin and DMT all display increased affinity for a variety of dopamine receptors relative to 5-MeO-DMT

Compared to other psychedelics, like LSD, psylocibin or DMT, 5-MeO-DMT can be administered to patients, preferably using dosing schemes as described herein, without a significant risk of inducing mania or hypomania in a patient suffering from a mental or nervous system disorder, including a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; a Psychotic Disorder, such as Schizophrenia; or a personality disorder, such as Schizotypal Personality Disorder. The patient suffering from such a mental or nervous system disorder, treated according to the invention, does not experience treatment-emergent mania or hypomania.

It is also noted that reports of treatment-emergent mania or hypomania related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.” •Journal of Psychiatric Practice•13.4 (2007): 233-237). However, the recently concluded clinical trial of 5-MeO-DMT in TRD patients showed no evidence of hypomania induction.

5-MeO-DMT can induce peak experiences, i.e., experiences characterized by an emotional perspective shift, which is described as “loss of ego” which often culminates in an overwhelming sense of “oneness with the universe”, more rapidly than other psychedelics and has a short duration of acute psychedelic effects (5 to 30 minutes after inhalation compared with several hours for e.g. oral psilocybin and oral LSD). These characteristics of 5-MeO-DMT are associated with an improved therapeutic profile which can be explained by specific alterations of Resting State Network (RSN) activity under 5-MeO-DMT treatment.

Furthermore, 5-MeO-DMT is a 5-HT7 receptor agonist showing high affinity towards the receptor. The inventors determined, using recombinant human 5-HT7 receptor, [³H]LSD as a radio ligand and serotonin to estimate non-specific binding, a Kᵢ of 2.3 nM.

Thus, besides the 5-HT1A and 5-HT2A receptors discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatal complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, psychomotor retardation, negative thinking or social/emotional withdrawal. This is supported by the clinical results demonstrated in studies referred to herein.

Another feature of 5-MeO-DMT is its short half-life.

5-MeO-DMT is mainly inactivated through a deamination pathway mediated by monoamine oxidase A, and it is O-demethylated by cytochrome P450 2D6 (CYP2D6) enzyme.

The inventors investigated pharmacokinetic properties of 5-MeO-DMT and observed rapid absorption and distribution of inhaled 5-MeO-DMT, with maximum concentrations and pharmacological effects observed during and immediately after dosing.

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations, for instance, 10 minutes, 2 hours, or 3 hours after administration.

The inventors have further determined that 5-MeO-DMT offers various characteristics that renders it an attractive treatment for PPD. In contrast to SSRIs, it is a rapid-acting agent (in a 5-MeO-DMT-TRD trial, 5/8 patients with TRD achieved a remission within 2 h after dosing, and 8/8 patients achieved a remission on day 1, with 7/8 patients maintaining their remission at Day 7). Using 5-MeO-DMT to treat PPD patients, not only can a rapid improvement of depressive symptoms be achieved, but also a rapid improvement of maternal functioning. Furthermore, 5-MeO-DMT is administered during a single-day treatment session, with optional infrequent redosing, thus differentiating it from SSRIs, which require a chronic daily dosing regimen associated with low compliance, and from brexanolone, requiring protracted infusions and hospital admission.

The present invention thus also addresses compliance and patient convenience. Furthermore, the inventors determined that a treatment of PPD with 5-MeO-DMT or a pharmaceutically acceptable salt thereof allows continuing breastfeeding with only a short interruption for the treatment.

According to the invention, isotopic variants of 5-MeO-DMT and pharmaceutically acceptable salts thereof can also be used. When reference is made to the use of 5-MeO-DMT or a pharmaceutically acceptable salt thereof, the use of isotopic variants is also contemplated.

These variants are in particular deuterated forms of 5-MeO-DMT and pharmaceutically acceptable salts of such forms.

Deuterated forms of 5-MeO-DMT are forms having a higher deuterium content than expected based on the natural abundance of this isotope.

Deuterated forms of 5-MeO-DMT are in particular forms wherein deuterium has been introduced at one or more defined hydrogen positions.

Examples of deuterated forms of 5-MeO-DMT include, without limitation, 1-deuterio-2-(5-methoxy-1H-indol-3-yl)-N,N-dimethylethanamine, 1,1-dideuterio-2-(5-methoxy-1H-indol-3-yl)-N,N-dimethylethanamine, 1,1,2,2-tetradeuterio-2-(5-methoxy-1H-indol-3-yl)-N,N-dimethylethanamine, and N,N-dimethyl-2-[5-(trideuteriomethoxy)-1H-indol-3-yl]ethanamine.

Further examples include forms of 5-MeO-DMT wherein deuterium has been introduced at one or more hydrogen positions of the N-bound methyl groups. Still further examples include forms of 5-MeO-DMT wherein one or more deuterium atoms replace hydrogen atoms of the indole ring system. It is moreover noted that combinations of the above substitution patterns are also contemplated.

Preparation methods for these compounds are known in the art.

According to the invention, mixtures of deuterated forms of 5-MeO-DMT, mixtures of one or more deuterated form with non-deuterated 5-MeO-DMT, pharmaceutically acceptable salts of deuterated forms of 5-MeO-DMT, mixture of such salts as well as mixtures of salts of deuterated and non-deuterated 5-MeO-DMT can also be used.

Further according to the invention, deuterated 5-MeO-DMT and salts of deuterated 5-MeO-DMT are used in amounts that are equimolar to the amounts of the corresponding non-deuterated forms.

According to the invention, prodrugs of 5-MeO-DMT and pharmaceutically acceptable salts of such prodrugs can also be used. Such prodrugs of 5-MeO-DMT can be metabolically converted to 5-MeO-DMT. Thus, when reference is made to the use of 5-MeO-DMT or a pharmaceutically acceptable salt thereof, this can be replaced by a 5-MeO-DMT prodrug or a salt thereof.

In suitable prodrugs, the hydrogen in position 1 of the indole moiety is substituted by an organic moiety which can be split off after administration.

Examples of suitable organic moieties are —C(O)OR¹, —C(O)R², —CH(R³)OR⁴, —C(O)OCH(R³)OC(O)R⁴, —C(O)OCH(R³)OC(O)OR⁴, —CH(R³)C(O)R⁴, —CH(R³)OC(O)R⁴, —CH(R³)OC(O)OR⁴, wherein each of R¹, R², R³, and R⁴ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently substituted or unsubstituted.

Preferred examples of organic moieties are —CH(R³)OC(O)R⁴ and —C(O)OR¹, wherein R¹, R³, and R⁴ are defined as above.

Prodrugs, especially those of the above structure, can also be used on the form of pharmaceutically acceptable salts.

Specific examples of prodrugs are 5-MeO-DMT carboxy-isopropyl valinate, preferably in salt form, in particular as ditrifluoroacetate (1-(((S)-2-amino-3-methylbutanoyl)oxy)-2-methylpropyl 3-(2-(dimethylamino)ethyl)-5-methoxy-1H-indole-1-carboxylate di-trifluoro-acetate) and 5-MeO-DMT methyl pivalate (3-(2-(dimethylamino)ethyl)-5-methoxy-1H-indol-1-yl)methyl pivalate).

Preparation methods for prodrugs as discussed herein are known in the art.

According to the invention, the Tₘₐₓ value of the metabolite 5-MeO-DMT as measured in male Sprague-Dawley (SD) rats following oral dosing of the prodrug at 10 mg/kg is preferably 1 hour or less, more preferably 0.7 hours or less and in particular 0.5 hours or less.

Further according to the invention, prodrugs of 5-MeO-DMT and salts of prodrugs of 5-MeO-DMT are used in amounts that are equimolar to the amounts of the corresponding non-prodrug forms.

### Treatment Aspects

As already indicated above, the present invention allows treating patients suffering from PPD. The treatment does not only lead to reductions in scores assessing the severity of depression, but also improves maternal functioning as discussed in detail below.

To further support the clinical application of 5-MeO-DMT in patients suffering from PPD the inventors assessed clinical data relating to the use of 5-MeO-DMT in patients treated because of mental disease and noted particular improvements in disease aspects typically also observed in patients with PPD. The inventors in particular noted improvements in various symptoms and combinations of symptoms which the inventors determined to be also associated with maternal functioning.

The data stem from a recently completed clinical trial investigating the use of 5-MeO-DMT in the treatment of patients diagnosed with Treatment Resistant Depression (TRD; see also the examples section below). While the completed trial did not involve patients suffering from PPD, the inventors determined, as discussed in detail below, that certain clinical observations are made in the trial are relevant for devising a treatment for PPD.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery Åsberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that several of the subcore items are of particular relevance for PPD patients and are related to maternal functioning.

Multiple patients within the recruited cohort displayed significant improvements in one or more of these subscore items, a result that confirms the inventors' finding that 5-MeO-DMT is a compound suitable for treating PPD patients and for improving maternal functioning in those patients.

The specific subscore items in each of the scales are identified in more detail below. The inventors conclude that efficacy in treating one or more of these symptoms will result in significant improvements in overall outcomes in PPD patients treated using 5-MeO-DMT.

Thus, a treatment according to the invention reduces or eliminates (or improves or eliminates) an aspect of the disease.

If the aspect is assessed on the MADRS scale, there is an improvement by at least one point (reduction) or the patient is in complete remission after the treatment (elimination), i.e., the respective aspect is scored 0.

If the aspect is assessed on the BPRS scale, there is an improvement by at least one point (reduction) or the patient is in complete remission after the treatment (elimination), i.e., the respective aspect is scored 1.

A clinical response may also be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

The inventors further consider that improvements observed in certain MADRS items will translate into improvements in aspects of maternal functioning.

MADRS items of particular relevance are discussed in more detail below.

The MADRS item “inner tension” represents feelings of ill-defined discomfort, edginess, inner turmoil, mental tension mounting to either panic, dread or anguish. It is rated according to intensity, frequency, duration and the extent of reassurance called for.

A score of 0 is assigned if the patient is placid and there is only fleeting inner tension. A score of 2 is assigned if there are occasional feelings of edginess and ill defined discomfort. The score is 4 if there are continuous feelings of inner tension or intermittent panic which the patient can only master with some difficulty. The score is 6 in case of unrelenting dread or anguish and overwhelming panic.

The inventors have determined that increases in the score of the MADRS item “inner tension” have negative impacts on both aspects of maternal functioning (maternal competence relating to interactions with the infant(s) as well as maternal self-care). Increased scores in the MADRS item “inner tension” impair mother-child interaction as well as psychological well-being of the mother as assessed by the BIMF.

Conversely, improvements regarding this MADRS item lead to improvements in maternal functioning, in particular in the BIMF functional domains mother-child interaction and/or psychological well-being of the mother.

In the above indicated trial involving TRD patients, in the study group receiving the individualized dosing regimen, the aggregated score for the MADRS item “inner tension” across all 8 patients was 26 at base line. After 2 hours, it was reduced to 11 which corresponds to an improvement of 15 points or 58%. At day 1 after treatment, it was reduced to 6 which corresponds to an improvement of 20 points or 77%. At day 7 after treatment, it was reduced to 12 which corresponds to an improvement of 14 points or 54%.

In the 12 mg group, the aggregated score for the MADRS item “inner tension” across all 4 patients was 13 at base line. After 2 hours, it was reduced to 2 which corresponds to an improvement of 11 points or 85%. At day 1 after treatment, it was reduced to 3 which corresponds to an improvement of 10 points or 77%. At day 7 after treatment, it was reduced to 5 which corresponds to an improvement of 8 points or 62%.

The inventors conclude that 5-MeO-DMT can be used to treat PPD patients to achieve a reduction or elimination of inner tension.

An improvement in inner tension is reflected by at least an improvement in the score of the MADRS item inner tension about 2 hours; on day 1, for instance, after about 24 hours; on day 7; on day 14; and/or on day 28 after the last administration of 5-MeO-DMT or a pharmaceutically acceptable salt thereof.

An improvement in inner tension as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in inner tension, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in inner tension, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of inner tension achieved by treating a PPD patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since inner tension also affects other aspects of PPD, the inventors conclude that the observed improvement in the “inner tension” item on the MADRS will additionally contribute to an overall improvement in maternal functioning.

The MADRS item “lassitude” represents a difficulty getting started or slowness initiating and performing everyday activities.

A score of 0 means that there is hardly any difficulty in getting started and no sluggishness. A score of 2 is assigned if the patient has difficulties in starting activities. A score of 4 means difficulties in starting simple routine activities which are carried out with effort.

A score of 6 is assigned in case of complete lassitude, the patient being unable to do anything without help.

The inventors have determined that increases in the score of the MADRS item “lassitude” have negative impacts on both aspects of maternal functioning (maternal competence relating to interactions with the infant(s) as well as maternal self-care). Increased scores in the MADRS item “lassitude” impair infant care, self-care, psychological well-being, management and adjustment.

Conversely, improvements regarding this MADRS item lead to improvements in maternal functioning, in particular in the BIMF functional domains infant care, self-care, psychological well-being, management and/or adjustment.

In the study group receiving the individualized dosing regimen, the aggregated score for the MADRS item “lassitude” across all 8 patients was 27 at base line. After 2 hours, it was reduced to 10 which corresponds to an improvement of 17 points or 63%. At day 1 after treatment, it was reduced to 5 which corresponds to an improvement of 22 points or 81%. At day 7 after treatment, it was reduced to 3 which corresponds to an improvement of 24 points or 89%.

In the 12 mg group, the aggregated score for the MADRS item “lassitude” across all 4 patients was 16 at base line. After 2 hours, it was reduced to 10 which corresponds to an improvement of 6 points or 38%. At day 1 after treatment, it was reduced to 0 which corresponds to an improvement of 16 points or 100%. At day 7 after treatment, it was reduced to 3 which corresponds to an improvement of 13 points or 81%.

The inventors conclude that 5-MeO-DMT can be used to treat PPD patients to achieve a reduction or elimination of lassitude.

An improvement in lassitude is reflected by at least an improvement in the score of the MADRS item lassitude about 2 hours; on day 1, for instance, after about 24 hours; on day 7; on day 14; and/or on day 28 after the last administration of 5-MeO-DMT or a pharmaceutically acceptable salt thereof.

An improvement in lassitude as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in lassitude, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in lassitude, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof. The inventors furthermore conclude that a reduction or elimination of lassitude achieved by treating a PPD patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof. Since lassitude also affects other aspects of PPD, the inventors conclude that the observed improvement in the “lassitude” item on the MADRS will additionally contribute to an overall improvement in maternal functioning.

The MADRS item “inability to feel” represents the subjective experience of reduced interest in the surroundings, or activities that normally give pleasure. The ability to react with adequate emotion to circumstances or people is reduced.

A score of 0 indicates normal interest in the surroundings and in other people, a score of 2 reduced ability to enjoy usual interests. A score of 4 is assigned in case of a loss of interest in the surroundings and a loss of feelings for friends and acquaintances. A score of 6 reflects the experience of being emotionally paralysed, inability to feel anger, grief or pleasure and a complete or even painful failure to feel for close relatives and friends.

The inventors have determined that increases in the score of the MADRS item “inability to feel” have negative impacts on both aspects of maternal functioning (maternal competence relating to interactions with the infant(s) as well as maternal self-care). Increased scores in the MADRS item “inability to feel” impair mother-child interaction and psychological well-being.

Conversely, improvements regarding this MADRS item lead to improvements in maternal functioning, in particular in the BIMF functional domains mother-child interaction and/or psychological well-being.

In the study group receiving the individualized dosing regimen, the aggregated score for the MADRS item “inability to feel” across all 8 patients was 36 at base line. After 2 hours, it was reduced to 12 which corresponds to an improvement of 24 points or 67%. At day 1 after treatment, it was reduced to 2 which corresponds to an improvement of 34 points or 94%. At day 7 after treatment, it was reduced to 6 which corresponds to an improvement of 30 points or 83%.

In the 12 mg group, the aggregated score for the MADRS item “inability to feel” across all 4 patients was 16 at base line. After 2 hours, it was reduced to 9 which corresponds to an improvement of 7 points or 44%. At day 1 after treatment, it was reduced to 1 which corresponds to an improvement of 15 points or 94%. At day 7 after treatment, it was reduced to 1 which corresponds to an improvement of 15 points or 94%.

The inventors conclude that 5-MeO-DMT can be used to treat PPD patients to achieve a reduction or elimination of inability to feel.

An improvement in inability to feel is reflected by at least an improvement in the score of the MADRS item inability to feel about 2 hours; on day 1, for instance, after about 24 hours; on day 7; on day 14; and/or on day 28 after the last administration of 5-MeO-DMT or a pharmaceutically acceptable salt thereof.

An improvement in inability to feel as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in inability to feel, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in inability to feel, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of inability to feel by treating a PPD patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 7; on day 14; and/or on day 28 after the last administration of 5-MeO-DMT or a pharmaceutically acceptable salt thereof 0.13 Since inability to feel also affects other aspects of PPD, the inventors conclude that the observed improvement in the “inability to feel” item on the MADRS will additionally contribute to an overall improvement in maternal functioning.

The MADRS item “concentration difficulties” represents difficulties in collecting one's thoughts mounting to incapacitating lack of concentration.

The score is 0 if the patient has no difficulties in concentrating. The score is 2 in case of occasional difficulties in collecting one's thoughts. A score of 4 is assigned in case of difficulties in concentrating and sustaining thought which reduces ability to read or hold a conversation. The score is 6 if the patient is unable to read or converse without great difficulty.

The inventors have determined that increases in the score of the MADRS item “concentration difficulties” have negative impacts on both aspects of maternal functioning (maternal competence relating to interactions with the infant(s) as well as maternal self-care).

Increased scores in the MADRS item “concentration difficulties” impair infant care as well as management.

Conversely, improvements regarding this MADRS item lead to improvements in maternal functioning, in particular in the BIMF functional domains infant care and/or management.

In the study group receiving the individualized dosing regimen, the aggregated score for the MADRS item “concentration difficulties” across all 8 patients was 30 at base line. After 2 hours, it was reduced to 11 which corresponds to an improvement of 19 points or 63%. At day 1 after treatment, it was reduced to 1 which corresponds to an improvement of 29 points or 97%. At day 7 after treatment, it was reduced to 9 which corresponds to an improvement of 21 points or 70%.

In the 12 mg group, the aggregated score for the MADRS item “concentration difficulties” across all 4 patients was 16 at base line. After 2 hours, it was reduced to 7 which corresponds to an improvement of 9 points or 56%. At day 1 after treatment, it was reduced to 2 which corresponds to an improvement of 14 points or 88%. At day 7 after treatment, it was reduced to 3 which corresponds to an improvement of 13 points or 81%.

The inventors conclude that 5-MeO-DMT can be used to treat PPD patients to achieve a reduction or elimination of concentration difficulties.

An improvement in concentration difficulties is reflected by at least an improvement in the score of the MADRS item concentration difficulties about 2 hours; on day 1, for instance, after about 24 hours; on day 7; on day 14; and/or on day 28 after the last administration of 5-MeO-DMT or a pharmaceutically acceptable salt thereof.

An improvement in concentration difficulties as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in concentration difficulties, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in concentration difficulties, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of concentration difficulties by treating a PPD patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 7; on day 14; and/or on day 28 after the last administration of 5-MeO-DMT or a pharmaceutically acceptable salt thereof.

Since concentration difficulties also affect other aspects of PPD, the inventors conclude that the observed improvement in the “concentration difficulties” item on the MADRS will additionally contribute to an overall improvement in maternal functioning.

The MADRS item “pessimistic thoughts” represents thoughts of guilt, inferiority, self-reproach, sinfulness, remorse and ruin.

A score of 0 is assigned if there are no pessimistic thoughts. The score is 2 in case of fluctuating ideas of failure, self-reproach or self-depreciation. A score means persistent self-accusations, or definite but still rational ideas of guilt or sin as well as the patient being increasingly pessimistic about the future. A score of 6 is assigned in case of delusions of ruin, remorse or unredeemable sin and self-accusations which are absurd and unshakable.

The inventors have determined that increases in the score of the MADRS item “pessimistic thoughts” have negative impacts on both aspects of maternal functioning (maternal competence relating to interactions with the infant(s) as well as maternal self-care). Increased scores in the MADRS item “pessimistic thoughts” impair psychological well-being, social support and management.

Conversely, improvements regarding this MADRS item lead to improvements in maternal functioning, in particular in the BIMF functional domains psychological wellbeing, social support and/or management.

In the study group receiving the individualized dosing regimen, the aggregated score for the MADRS item “pessimistic thoughts” across all 8 patients was 28 at base line. After 2 hours, it was reduced to 7 which corresponds to an improvement of 21 points or 75%. At day 1 after treatment, it was reduced to 4 which corresponds to an improvement of 24 points or 86%. At day 7 after treatment, it was reduced to 3 which corresponds to an improvement of 25 points or 89%.

In the 12 mg group, the aggregated score for the MADRS item “pessimistic thoughts” across all 4 patients was 16 at base line. After 2 hours, it was reduced to 8 which corresponds to an improvement of 8 points or 50%. At day 1 after treatment, it was reduced to 7 which corresponds to an improvement of 9 points or 56%. At day 7 after treatment, it was reduced to 8 which corresponds to an improvement of 8 points or 50%.

The inventors conclude that 5-MeO-DMT can be used to treat PPD patients to achieve a reduction or elimination of pessimistic thoughts.

An improvement in pessimistic thoughts is reflected by at least an improvement in the score of the MADRS item pessimistic thoughts about 2 hours; on day 1, for instance, after about 24 hours; on day 7; on day 14; and/or on day 28 after the last administration of 5-MeO-DMT or a pharmaceutically acceptable salt thereof.

An improvement in pessimistic thoughts as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in pessimistic thoughts, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in pessimistic thoughts, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of pessimistic thoughts by treating a PPD patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since pessimistic thoughts also affects other aspects of PPD, the inventors conclude that the observed improvement in the “pessimistic thoughts” item on the MADRS will additionally contribute to an overall improvement in maternal functioning.

The MADRS item “reduced sleep” represents the experience of reduced duration or depth of sleep compared to the subject's own normal pattern when well.

A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

The inventors have determined that increases in the score of the MADRS item “reduced sleep” have negative impacts on both aspects of maternal functioning (maternal competence relating to interactions with the infant(s) as well as maternal self-care). Increased scores in the MADRS item “reduced sleep” impair self-care, psychological well-being and management.

Conversely, improvements regarding this MADRS item lead to improvements in maternal functioning, in particular in the BIMF functional domains self-care, psychological well-being and/or management.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

The inventors conclude that 5-MeO-DMT can be used to treat PPD patients to achieve a reduction or elimination of reduced sleep.

The reduction or elimination of reduced sleep is reflected by at least an improvement in the score of the MADRS item reduced sleep on day 1, for instance, after about 24 hours; on day 7; on day 14; and/or on day 28 after the last administration of 5-MeO-DMT or a pharmaceutically acceptable salt thereof.

An improvement in reduced sleep as reflected by a reduction in the Clinical Global Impression-Severity (CGI-S) score, occurs not later than about 24 hours after the last administration of 5-MeO-DMT or a pharmaceutically acceptable salt thereof.

An improvement in reduced sleep, as reflected by at least a score of “much improved” in the Clinical Global Impression-Improvement (CGI-I) score or the Patient Global Impression-Improvement (PGI-I) score, preferably occurs not later than about 24 hours after the last administration of 5-MeO-DMT or a pharmaceutically acceptable salt thereof.

An improvement in reduced sleep, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of reduced sleep by treating a PPD patient does not only lead to a reduction in the MADRS total score, but also to an improvement in maternal functioning, as reflected by an increase in the BIMF score. This improvement will be achieved rapidly, namely within about 24 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since reduced sleep also affects other aspects of PPD, the inventors conclude that the observed improvement in the “reduced sleep” item on the MADRS will additionally contribute to an overall improvement in maternal functioning.

A further aspect of PPD which can be treated by administration of 5-MeO-DMT, is suicidal ideation. 5-MeO-DMT can be administered to PPD patients to reduce or eliminate suicidal ideation in said patients.

In the above-mentioned clinical studies involving the administration of 5-MeO-DMT, among others the MADRS item “suicidal thoughts” was assessed.

“Suicidal thoughts” represents a feeling that life is not worth living, that a natural death would be welcome, having suicidal thoughts, and/or making the preparations for suicide. Suicidal attempts should not in themselves influence the rating for this MADRS item.

A score of 0 means that the patient enjoys life. A score of 2 is assigned if the PPD patient is weary of life, and/or has only fleeting suicidal thoughts. A score of 4 means the patient feels they would be better off dead, suicidal thoughts are common and suicide is considered as a possible solution but the patient has no specific plans or intention. A score of 6 is assigned in case the patient has explicit plans for suicide and/or is making active preparations.

This MADRS scale item is of particular relevance to suicidal ideation.

The inventors have determined that increases in the score of the MADRS item “suicidal thoughts” have negative impacts on both aspects of maternal functioning (maternal competence relating to interactions with the infant(s) as well as maternal self-care). Increased scores in the MADRS item “suicidal thoughts” impair self-care, psychological well-being and management.

Conversely, improvements regarding this MADRS item lead to improvements in maternal functioning, in particular in the BIMF functional domains self-care, psychological well-being and/or management.

In the study group receiving the individualized dosing regimen, the aggregated score for the MADRS item “suicidal thoughts” across all 8 patients was 11 at base line. After 2 hours, it was reduced to 3 which corresponds to an improvement of 8 points or 73%. At day 1 after treatment, it was reduced to 1 which corresponds to an improvement of 10 points or 91%. At day 7 after treatment, it was reduced to 3 which corresponds to an improvement of 8 points or 73%.

In the 12 mg group, the aggregated score for the MADRS item “suicidal thoughts” across all 4 patients was 8 at base line. After 2 hours, it was reduced to 3 which corresponds to an improvement of 5 points or 63%. At day 1 after treatment, it was reduced to 5 which corresponds to an improvement of 3 points or 38%. At day 7 after treatment, it was reduced to 7 which corresponds to an improvement of 1 point or 13%.

Thus, the score of the scale item that is of particular relevance to suicidal ideation, “suicidal thoughts”, is markedly improved, at least in the individualized dosing regimen patients. The inventors conclude that 5-MeO-DMT can be used to treat suicidal ideation in PPD patients.

Thus, according to the invention, the treatment of a PPD patient suffering from suicidal ideation reduces or eliminates the suicidal ideation.

The reduction or elimination of suicidal ideation is reflected by at least an improvement in the score of the MADRS item suicidal thoughts about 2 hours; on day 1, for instance, after about 24 hours, on day 7; on day 14; and/or on day 28 after the last administration of 5-MeO-DMT or a pharmaceutically acceptable salt thereof.

If the patient suffers from suicidal ideation the improvement in suicidal ideation is reflected by a reduction in the Clinical Global Impression-Severity (CGI-S) score about 2 hours; on day 1, for instance, after about 24 hours; on day 7; on day 14; and/or on day 28 after the last administration of 5-MeO-DMT or a pharmaceutically acceptable salt thereof.

An improvement in suicidal ideation as reflected by a reduction in the Clinical Global Impression-Severity (CGI-S) score, occurs not later than about 2 hours after the last administration of 5-MeO-DMT or a pharmaceutically acceptable salt thereof.

Alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in suicidal ideation, as assessed by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in suicidal ideation, as assessed by a reduction of the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of suicidal thoughts by treating a PPD patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since suicidal thoughts also affects other aspects of PPD, the inventors conclude that the observed improvement in the “suicidal thoughts” item on the MADRS will additionally contribute to an overall improvement in maternal functioning.

The BPRS item “emotional withdrawal” relates to a deficiency in the patient's ability to relate emotionally during the interview situation. Possible scores are:
1—No emotional withdrawal.
2—Very Mild. Lack of emotional involvement shown by occasional failure to make reciprocal comments, occasionally appearing preoccupied, or smiling in a stilted manner, but spontaneously engages the interviewer most of the time.
3—Mild. Lack of emotional involvement shown by noticeable failure to make reciprocal comments, appearing preoccupied, or lacking in warmth, but responds to interviewer when approached.
4—Moderate. Emotional contact not present much of the interview because subject does not elaborate responses, fails to make eye contact, doesn't seem to care if interviewer is listening, or may be preoccupied with psychotic material.
5—Moderately Severe. Same as “4” but emotional contact not present most of the interview.
6—Severe. Actively avoids emotional participation. Frequently unresponsive or responds with yes/no answers (not solely due to persecutory delusions). Responds with only minimal affect.
7—Extremely Severe. Consistently avoids emotional participation. Unresponsive or responds with yes/no answers (not solely due to persecutory delusions). May leave during interview or just not respond at all.

The inventors have determined that increases in the score of the BPRS item “emotional withdrawal” have negative impacts on both aspects of maternal functioning (maternal competence relating to interactions with the infant(s) as well as maternal self-care). Increased scores in the BPRS item “emotional withdrawal” impair psychological well-being, mother-child interaction and social support.

Conversely, improvements regarding this BPRS item lead to improvements in maternal functioning, in particular in the BIMF functional domains psychological well-being, mother-child interaction and/or social support.

In the study group receiving the individualized dosing regimen, aggregated score for the BPRS item “emotional withdrawal” was 13 at base line. After 3 hours, it was reduced to 8 which corresponds to an improvement of 5 points or 38%. At day 1 after treatment, it was reduced to 8 which corresponds to an improvement of 5 points or 38%. At day 7 after treatment, it was reduced to 8 which corresponds to an improvement of 5 points or 38%.

In the 12 mg group, the aggregated score for the BPRS item “emotional withdrawal” was 13 at base line. After 3 hours, it was reduced to 11 which corresponds to an improvement of 2 points or 15%. At day 1 after treatment, it was reduced to 8 which corresponds to an improvement of 5 points or 38%. At day 7 after treatment, it was reduced to 6 which corresponds to an improvement of 7 points or 54%.

The inventors conclude that 5-MeO-DMT can be used to treat PPD patients to achieve a reduction or elimination of emotional withdrawal.

The reduction or elimination of emotional withdrawal is reflected by at least an improvement in the score of the BPRS item emotional withdrawal about 2 hours; on day 1, for instance, after about 24 hours; on day 7; on day 14; and/or on day 28 after the last administration of 5-MeO-DMT or a pharmaceutically acceptable salt thereof.

An improvement in emotional withdrawal as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in emotional withdrawal, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in emotional withdrawal, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of emotional withdrawal by treating a PPD patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since emotional withdrawal also affects other aspects of PPD, the inventors conclude that the observed improvement in the “emotional withdrawal” item on the BPRS will additionally contribute to an overall improvement in maternal functioning.

The BPRS item “blunted affect” relates to a restricted range in emotional expressiveness of face, voice, and gestures as well as a marked indifference or flatness even when discussing distressing topics. Possible scores are:
1—No blunted affect.
2—Very Mild. Emotional range is slightly subdued or reserved but displays appropriate facial expressions and tone of voice that are within normal limits.
3—Mild. Emotional range overall is diminished, subdued, or reserved, without many spontaneous and appropriate emotional responses. Voice tone is slightly monotonous.
4—Moderate. Emotional range is noticeably diminished, patient doesn't show emotion, smile, or react to distressing topics except infrequently. Voice tone is monotonous or there is noticeable decrease in spontaneous movements. Displays of emotion or gestures are usually followed by a return to flattened affect.
5—Moderately Severe. Emotional range very diminished, patient doesn't show emotion, smile or react to distressing topics except minimally, few gestures, facial expression does not change very often. Voice tone is monotonous much of the time.
6—Severe. Very little emotional range or expression. Mechanical in speech and gestures most of the time. Unchanging facial expression. Voice tone is monotonous most of the time.
7—Extremely Severe. Virtually no emotional range or expressiveness, stiff movements. Voice tone is monotonous all of the time.

The inventors have determined that increases in the score of the BPRS item “blunted affect” have negative impacts on both aspects of maternal functioning (maternal competence relating to interactions with the infant(s) as well as maternal self-care). Increased scores in the BPRS item “blunted affect” impair psychological well-being and mother-child interaction.

Conversely, improvements regarding this BPRS item lead to improvements in maternal functioning, in particular in the BIMF functional domains psychological well-being and/or mother-child interaction.

The aggregated score for the BPRS item “blunted affect” was 15 at base line. After 3 hours, it was reduced to 11 which corresponds to an improvement of 4 points or 27%. At day 1 after treatment, it was reduced to 8 which corresponds to an improvement of 7 points or 47%. At day 7 after treatment, it was reduced to 8 which corresponds to an improvement of 7 points or 47%.

In the 12 mg group, the aggregated score for the BPRS item “blunted affect” was 11 at base line. After 3 hours, it was reduced to 8 which corresponds to an improvement of 3 points or 27%. At day 1 after treatment, it was reduced to 6 which corresponds to an improvement of 5 points or 45%. At day 7 after treatment, it was reduced to 5 which corresponds to an improvement of 6 points or 55%.

The inventors conclude that 5-MeO-DMT can be used to treat PPD patients to achieve a reduction or elimination of blunted affect.

The reduction or elimination of blunted affect is reflected by at least an improvement in the score of the BPRS item blunted affect about 2 hours; on day 1, for instance, after about 24 hours; on day 7; on day 14; and/or on day 28 after the last administration of 5-MeO-DMT or a pharmaceutically acceptable salt thereof.

An improvement in blunted affect as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in blunted affect, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in blunted affect, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of blunted affect by treating a PPD patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since blunted affect also affects other aspects of PPD, the inventors conclude that the observed improvement in the “blunted affect” item on the BPRS will additionally contribute to an overall improvement in maternal functioning.

The BPRS item “guilt feelings” relates to over concern or remorse for past behavior. Possible scores are:
1—No guilt feelings.
2—Very Mild. Concerned about having failed someone or at something but not preoccupied. Can shift thoughts to other matters easily.
3—Mild. Concerned about having failed someone or at something with some preoccupation. Tends to voice guilt to others.
4—Moderate. Disproportionate preoccupation with guilt, having done wrong, injured others by doing or failing to do something, but can readily turn attention to other things.
5—Moderately Severe. Preoccupation with guilt, having failed someone or at something, can turn attention to other things, but only with great effort. Not delusional.
6—Severe. Delusional guilt or unreasonable self-reproach very out of proportion to circumstances. Moderate preoccupation present.
7—Extremely Severe. Delusional guilt or unreasonable self-reproach grossly out of proportion to circumstances. Subject is very preoccupied with guilt and is likely to disclose to others or act on delusions.

The inventors have determined that increases in the score of the BPRS item “guilt feelings” have negative impacts on both aspects of maternal functioning (maternal competence relating to interactions with the infant(s) as well as maternal self-care). Increased scores in the BPRS item “guilt feelings” impair self-care, mother-child interaction, psychological wellbeing and management.

Conversely, improvements regarding this BPRS item lead to improvements in maternal functioning, in particular in the BIMF functional domains self-care, mother-child interaction, psychological wellbeing and/or management.

In the study group receiving the individualized dosing regimen, the aggregated score for the BPRS item “guilt feelings” across all 8 patients was 34 at base line. After 3 hours, it was reduced to 14 which corresponds to an improvement of 20 points or 59%. At day 1 after treatment, it was reduced to 11 which corresponds to an improvement of 23 points or 68%. At day 7 after treatment, it was reduced to 10 which corresponds to an improvement of 24 points or 71%.

In the 12 mg group, the aggregated score for the BPRS item “guilt feelings” across all 4 patients was 18 at base line. After 3 hours, it was reduced to 9 which corresponds to an improvement of 9 points or 50%. At day 1 after treatment, it was reduced to 5 which corresponds to an improvement of 13 points or 72%. At day 7 after treatment, it was reduced to 5 which corresponds to an improvement of 13 points or 72%.

The inventors conclude that 5-MeO-DMT can be used to treat PPD patients to achieve a reduction or elimination of guilt feelings.

The reduction or elimination of guilt feelings is reflected by at least an improvement in the score of the BPRS item guilt feelings about 2 hours; on day 1, for instance, after about 24 hours; on day 7; on day 14; and/or on day 28 after the last administration of 5-MeO-DMT or a pharmaceutically acceptable salt thereof.

An improvement in guilt feelings as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in guilt feelings, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in guilt feelings, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of guilt feelings by treating a PPD patient does not only lead to a reduction in the BPRS total score, but also to an improvement in maternal functioning, as reflected by an increase in the BIMF score.

This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since guilt feelings also affects other aspects of PPD, the inventors conclude that the observed improvement in the “guilt feelings” item on the BPRS will additionally contribute to an overall improvement in maternal functioning.

The BPRS item “anxiety” relates to reported apprehension, tension, fear, panic or worry.

Possible scores are:
1—No anxiety.
2—Very Mild. Reports some discomfort due to worry or infrequent worries that occur more than usual for most normal individuals.
3—Mild. Worried frequently but can readily turn attention to other things.
4—Moderate. Worried most of the time and cannot turn attention to other things easily but no impairment in functioning or occasional anxiety with autonomic accompaniment but no impairment in functioning.
5—Moderately Severe. Frequent, but not daily, periods of anxiety with autonomic accompaniment or some areas of functioning are disrupted by anxiety or worry.
6—Severe. Anxiety with autonomic accompaniment daily but not persisting throughout the day or many areas of functioning are disrupted by anxiety or constant worry.
7—Extremely Severe. Anxiety with autonomic accompaniment persisting throughout the day or most areas of functioning are disrupted by anxiety or constant worry.

The inventors have determined that increases in the score of the BPRS item “anxiety” have negative impacts on both aspects of maternal functioning (maternal competence relating to interactions with the infant(s) as well as maternal self-care). Increased scores in the BPRS item “anxiety” impair psychological wellbeing, social support and management.

Conversely, improvements regarding this BPRS item lead to improvements in maternal functioning, in particular in the BIMF functional domains psychological wellbeing, social support and/or management.

In the study group receiving the individualized dosing regimen, the aggregated score for the BPRS item “anxiety” across all 8 patients was 37 at base line. After 3 hours, it was reduced to 19 which corresponds to an improvement of 18 points or 49%. At day 1 after treatment, it was reduced to 16 which corresponds to an improvement of 21 points or 57%. At day 7 after treatment, it was reduced to 17 which corresponds to an improvement of 20 points or 54%.

In the 12 mg group, the aggregated score for the BPRS item “anxiety” across all 4 patients was 25 at base line. After 3 hours, it was reduced to 11 which corresponds to an improvement of 14 points or 56%. At day 1 after treatment, it was reduced to 6 which corresponds to an improvement of 19 points or 76%. At day 7 after treatment, it was reduced to 6 which corresponds to an improvement of 19 points or 76%.

The inventors conclude that 5-MeO-DMT can be used to treat PPD patients to achieve a reduction or elimination of anxiety.

The reduction or elimination of anxiety is reflected by at least an improvement in the score of the BPRS item anxiety about 2 hours; on day 1, for instance, after about 24 hours; on day 7; on day 14; and/or on day 28 after the last administration of 5-MeO-DMT or a pharmaceutically acceptable salt thereof.

An improvement in anxiety as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in anxiety, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in anxiety, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of anxiety by treating a PPD patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since anxiety also affects other aspects of PPD, the inventors conclude that the observed improvement in the “anxiety” item on the BPRS will additionally contribute to an overall improvement in maternal functioning.

The BPRS item “tension” relates to observable physical and motor manifestations of tension, “nervousness,” and agitation. Possible scores are
1—No tension.
2—Very Mild. More fidgety than most but within normal range. A few transient signs of tension, e.g., picking at fingernails, foot wagging, scratching scalp several times, or finger tapping.
3—Mild. Same as “2,” but with more frequent or exaggerated signs of tension.
4—Moderate. Many and frequent signs of motor tension with one or more signs sometimes occurring simultaneously, e.g., wagging one's foot while wringing hands together. There are times when no signs of tension are present.
5—Moderately Severe. Many and frequent signs of motor tension with one or more signs often occurring simultaneously. There are still rare times when no signs of tension are present.
6—Severe. Same as “5,” but signs of tension are continuous.
7—Extremely Severe. Multiple motor manifestations of tension are continuously present, e.g., continuous pacing and hand wringing.

The inventors have determined that increases in the score of the BPRS item “tension” have negative impacts on both aspects of maternal functioning (maternal competence relating to interactions with the infant(s) as well as maternal self-care). Increased scores in the BPRS item “tension” impair mother-child interaction and psychological wellbeing.

Conversely, improvements regarding this BPRS item lead to improvements in maternal functioning, in particular in the BIMF functional domains mother-child interaction and/or psychological wellbeing.

In the study group receiving the individualized dosing regimen, the aggregated score for the BPRS item “tension” across all 8 patients was 16 at base line. After 3 hours, it was reduced to 11 which corresponds to an improvement of 5 points or 31%. At day 1 after treatment, it was reduced to 11 which corresponds to an improvement of 5 points or 31%. At day 7 after treatment, it was reduced to 10 which corresponds to an improvement of 6 points or 38%.

In the 12 mg group, the aggregated score for the BPRS item “tension” across all 4 patients was 14 at base line. After 3 hours, it was reduced to 9 which corresponds to an improvement of 5 points or 36%. At day 1 after treatment, it was reduced to 6 which corresponds to an improvement of 8 points or 57%. At day 7 after treatment, it was reduced to 6 which corresponds to an improvement of 8 points or 57%.

The inventors conclude that 5-MeO-DMT can be used to treat PPD patients to achieve a reduction or elimination of tension.

The reduction or elimination of tension is reflected by at least an improvement in the score of the BPRS item tension about 2 hours; on day 1, for instance, after about 24 hours; on day 7; on day 14; and/or on day 28 after the last administration of 5-MeO-DMT or a pharmaceutically acceptable salt thereof.

An improvement in tension as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in tension, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in tension, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of tension by treating a PPD patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since tension also affects other aspects of PPD, the inventors conclude that the observed improvement in the “tension” item on the BPRS will additionally contribute to an overall improvement in maternal functioning.

Improvements in one or more aspects of PPD will also lead to overall improvements. Preferably, treatment leads to a remission.

A remission of depressive symptoms may be reflected by a MADRS score equal to or less than 10 and occurs not later than about 2 hours; occurs on day 1, for instance, after about 24 hours; on day 7; on day 14 and/or on day 28 after the last administration of 5-MeO-DMT or a pharmaceutically acceptable salt thereof.

Further alternatively or in addition, a remission of depressive symptoms may be reflected by a HAM-D score equal to or less than 7 and occurs not later than about 2 hours; occurs on day 1, for instance, after about 24 hours; on day 7; on day 14 and/or on day 28 after the last administration of 5-MeO-DMT or a pharmaceutically acceptable salt thereof.

It follows from the above that treatment of PPD patients with 5-MeO-DMT or a pharmaceutically acceptable salt thereof does not only lead to a reduction of the MADRS score, including in particular the subscores as detailed above, but also to improvements in the domains of the BIMF scale. The reduction of the MADRS score as well as improvements in maternal functioning are confirmed by clinical data as discussed in the example section below.

Improvements in maternal functioning include improvements in the functional domain of self-care. For instance, improvements in the MADRS items lassitude and/or reduced sleep lead to an increase in the BIMF scale scores reflecting self-care. The improvement of the cumulative score of the BIMF scale items reflecting self-care is preferably at least 10%, more preferably at least 20%.

Improvements in maternal functioning include improvements in the functional domain of infant care. For instance, improvements in the MADRS items lassitude and/or concentration difficulties lead to an increase in the BIMF scale scores reflecting infant care. The improvement of the cumulative score of the BIMF scale items reflecting self-care is preferably at least 15%, more preferably at least 25%.

Improvements in maternal functioning include improvements in the functional domain of mother-child interaction. For instance, improvements in the MADRS items inability to feel and inner tension lead to an increase in the BIMF scale scores reflecting mother-child interaction. The improvement of the cumulative score of the BIMF scale items reflecting mother-child interaction is preferably at least 5%, more preferably at least 15%

Improvements in maternal functioning include improvements in the functional domain of psychological well-being. For instance, improvements in the MADRS items lassitude, pessimistic thoughts, inability to feel, inner tension and/or reduced sleep lead to an increase in the BIMF scale scores reflecting psychological well-being. The improvement of the cumulative score of the BIMF scale items reflecting psychological well-being is preferably at least 25%, more preferably at least 35%.

Improvements in maternal functioning include improvements in the functional domain of social support. For instance, improvements in the MADRS item pessimistic thoughts leads to an increase in the BIMF scale scores reflecting social support. The improvement of the cumulative score of the BIMF scale items reflecting social support is preferably at least 10%, more preferably at least 20%

Improvements in maternal functioning include improvements in the functional domain of management. For instance, improvements in the MADRS items lassitude, pessimistic thoughts and/or concentration difficulties lead to an increase in the BIMF scale scores reflecting management. The improvement of the cumulative score of the BIMF scale items reflecting management is preferably at least 20%, more preferably at least 30%

Improvements in maternal functioning include improvements in the functional domain of adjustment. For instance, improvements in the MADRS item lassitude leads to an increase in the BIMF scale scores reflecting adjustment. The improvement of the cumulative score of the BIMF scale items reflecting adjustment is preferably at least 5%, more preferably at least 15%

The improvement in maternal functioning relates to one or more, in particular two or more functional domains according to the Barkin Index of Maternal Functioning (BIMF) selected from self-care, infant care, mother-child interaction, psychological wellbeing of the mother, social support, management, and adjustment.

The BIMF total score is improved by 10% or more, preferably by 20% or more.

### Breast Feeding

As indicated above, for many medications, breastfeeding PPD patients may be confronted with a situation where a decision must be made whether to discontinue breast-feeding or to discontinue/abstain from therapy.

In case the decision is taken to discontinue breast-feeding in order to receive a treatment, this decision will have a negative impact on maternal functioning and in particular compromise the functional domains mother-child interaction and psychological well-being.

The present invention also addresses the need for treating PPD in a breastfeeding mother without substantial interruption of breastfeeding.

According to the invention, breastfeeding can be resumed shortly after the treatment.

The inventors have investigated pharmacokinetic properties and metabolization of 5-MeO-DMT in an effort to determine from which point in time onwards after administration of 5-MeO-DMT or of a pharmaceutically acceptable salt breastfeeding is possible without exposing the suckling child to any relevant risk.

Absorption and distribution of inhaled 5-MeO-DMT is rapid, with maximum concentrations and pharmacological effects observed during and immediately after dosing.

Plasma protein binding is low (13-23%).

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations, for instance, 10 minutes, 2 hours, or 3 hours after administration.

Metabolites of 5-MeO-DMT which may occur in humans were identified to assess the potential relevance of such metabolites. In an in vitro metabolism identification study of human hepatocytes, 5-MeO-DMT free base was incubated for up to 120 minutes at 10 μM. Compounds identified and their relative proportions are shown in Table 1 below:

TABLE 1

| Compound | Relative Proportion |
|---|---|
| Bufotenine (5-OH-DMT) | 5% |
| 5-MeO-DMT | 7% |
| 5-MeO-DMT-N-oxide | 1% |
| 5-Methoxyindole-3-ethanol (5-methoxy- | 25% |
| tryptophol) | |
| 5-Methoxyindole acetic acid (5-MIAA) | 61% |

It is noted that subsequent assays repeatedly failed to detect the presence of 5-methoxytryptophol but reproducibly indicated the presence of 5-MIAA as the predominant metabolite. 5-Methoxytryptophol is thus not likely to play any significant role in vivo.

Metabolites as listed in the above table are formed via three different pathways.

The two most significant metabolites, 5-methoxyindole acetic acid and 5-methoxyindole-3-ethanol, are formed via oxidative deamination. This involves enzymatic removal of the N-methyl groups and oxidation so that an acetaldehyde is formed:

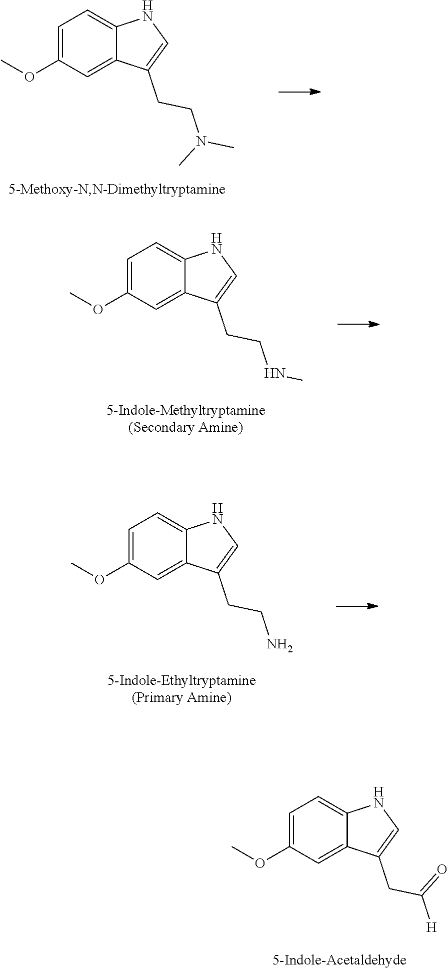

The indicated reaction is catalysed by monoamine oxidase A (MAO-A).

The secondary amine, the primary amine and the aldehyde were not identified which indicates that they are not present at any time in a significant concentration.

The aldehyde intermediate metabolite undergoes 2 separate biotransformations in human liver hepatocytes. It is either oxidised to 5-methoxyindole acetic acid or reduced to 5-methoxyindole-3-ethanol.

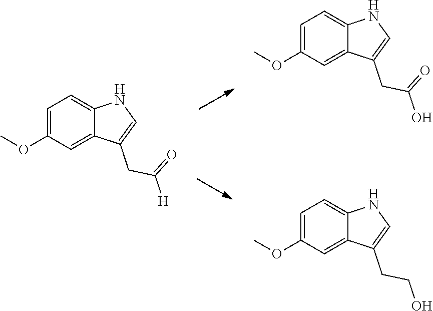

Both resulting metabolites are endogenous substances and are formed in the human body, for instance, during synthesis and metabolism of melatonin and serotonin (see e.g. Biochemistry of the Pineal. Chapter 3. in Melatonin and the Mammalian Pineal Gland. Arendt J (Ed.) Chapman & Hall, 1995; Slominski R and Slominski A T. Synthesis and Metabolism of Melatonin in the Skin and Retinal Pigment Epithelium. Chapter 3. in Melatonin in the Promotion of Health. Watson RR (Ed.) CRC Press 2012).

Since the predominant pathway of 5-MeO-DMT metabolization rapidly leads to metabolites that are also part of endogenous metabolic pathways, the inventors determined that the oxidative deamination of 5-MeO-DMT does not involve metabolites that would require imposing a limitation regarding breastfeeding.

Furthermore, as described in detail in the example section, incubation of 5-methoxytryptophol with human hepatocytes indicates a high metabolic turnover, with complete disappearance of the compound in 24 h. At 1 μM test concentration, in vitro intrinsic clearance for 5-methoxytryptophol was 16.2 μl/min/million cells (half-life 142 min).

Thus, the plasma concentration of 5-methoxytryptophol, to the extent it is formed at all, will rapidly decrease and reach endogenous levels.

5-MIAA is a weak acid, which will be present in plasma in ionized form, which decreases the likelihood of the compound entering into breast milk.

Incubation of 5-MIAA with human hepatocytes indicates a low metabolic turnover with, remaining 5-MIAA concentrations after 72 h being 75-82%. 5-MIAA is considered to be a final metabolite of 5-MeO-DMT.

5-MIAA shows relatively low plasma binding of ˜50% (mean fraction unbound (Fu); see the example section). It remains in circulation subject to renal clearance. Given a standard glomerular filtration rate of 90-120 ml/min, this means that all traces of 5-MIAA are removed from circulation for urinary excretion in approximately 1-2 hours (depending on patient size and taking into account the increase in blood volume that occurs during pregnancy).

In consequence, the plasma concentration of 5-MIAA will also rapidly decrease.

Coupled with the rapid clearance of 5-MeO-DMT (<1 hour), this supports the view that the administered therapy and all relevant metabolites have been cleared from circulation in approximately 2 hours.

A further metabolite identified, bufotenine, is the result of O-demethylation, which is catalysed by CYP2D6. The metabolized formed is then subject to glucuronidation, which is catalysed by UGT:

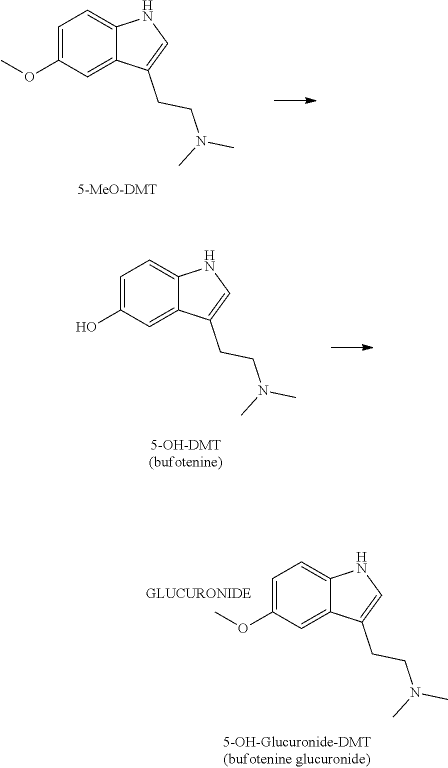

As part of a pharmacokinetic study, it was determined that bufotenine is barely detected in human serum. In no case is it detected 15 minutes after administration of 5-MeO-DMT. Bufotenine glucuronide cannot bind to receptors and does not exert any effect. Moreover, its concentration is so low that it was not detected in the hepatocyte assay. Bufotenine glucuronide is further converted to 5-hydroxyindole acetic acid:

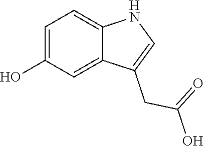 [SMILES: O=C(O)Cc1c[nH]c2ccc(O)cc12]

5-hydroxyindole acetic acid is an endogenous substance, for instance, it occurs in the metabolism of melatonin and serotonin (references as above).

Since the O-demethylation pathway of 5-MeO-DMT leads to a primary metabolite, bufotenine, which is rapidly cleared from the plasma and the further metabolization leads to compounds present only in very low concentration and ultimately to a metabolite that is also part of endogenous metabolic pathways, the inventors determined that the O-demethylation of 5-MeO-DMT does not involve metabolites that would require imposing a limitation regarding breastfeeding.

The third metabolic pathway involves N-oxidation:

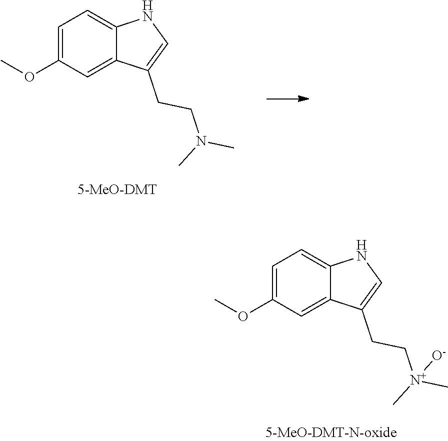

In silico modelling of the metabolite formed, 5-MeO-DMT-N-oxide was deemed to be non-genotoxic in line with the negative in vitro genotoxicity assessment of the parent molecule. The compound is water soluble and subject to rapid excretion, as confirmed by observations in the rat (Sitaram, B. R., Lockett, L., Blackman, G. L., McLeod, W. R., 1987. Urinary excretion of 5-methoxy-N,N-dimethyltryptamine, N,N-dimethyltryptamine and their N-oxides in the rat. Biochemical Pharmacology 36: 2235-2231). Since the pathway of 5-MeO-DMT metabolization involving N-oxidation plays only a minor role and leads to a low proportion of a metabolite without apparent toxicity which is rapidly excreted, the inventors determined that the N-oxidation of 5-MeO-DMT does not involve metabolites that would require imposing a limitation regarding breastfeeding.

Based on the above, the inventors have determined that breast feeding can be resumed shortly after the treatment with 5-MeO-DMT or a pharmaceutically acceptable salt thereof.

Thus, in case the patient is a breastfeeding mother she is advised to discontinue breast-feeding until 48 hours after the last administration of 5-MeO-DMT or a pharmaceutically acceptable salt thereof. In particular, she is advised to discontinue breastfeeding until 24 hours after the last administration of 5-MeO-DMT or a pharmaceutically acceptable salt thereof.

Preferably, breastfeeding has to be interrupted for only 6 hours, more preferably, for only 3 hours, most preferably for only 2 hours after the last administration of 5-MeO-DMT or a pharmaceutically acceptable salt thereof.

This short interruption and the corresponding possibility to resume breastfeeding shortly after treatment contributes to treatment success and in particular to maternal functioning and well-being and development of the infant(s).

### Modes of Administration

The therapeutically effective amount of 5-MeO-DMT is administered by inhalation, by nasal administration, by buccal administration or by sublingual administration. Administration via these routes can assure a rapid onset of action. A most preferred route of administration is administration by inhalation. Preferably, the inhalation of the therapeutically effective amount of 5-MeO-DMT occurs within a single breath.

For nasal administration, 5-MeO-DMT can be employed as a neat substance or in the form of a formulation for nasal administration, examples of which are known in the art. For nasal administration, 5-MeO-DMT can be employed as a pharmaceutically acceptable salt, preferably the hydrobromide salt, or in the form of a formulation of a pharmaceutically acceptable salt, preferable the hydrobromide salt. Examples of appropriate devices are known in the art.

Buccal administration or sublingual administration can also rely on a pharmaceutically acceptable salt of 5-MeO-DMT, preferable the hydrobromide salt, as such or in the form of formulations, for instance, tablets, films, sprays, creams, as generally known in the art.

Administration is in particular by inhalation of an aerosol. Such an aerosol comprises (a) a pharmaceutically acceptable gas; (b) aerosol particles of 5-methoxy-N,N-dimethyltryptamine (5-MeO-DMT) or a pharmaceutically acceptable salt thereof, wherein the aerosol has an aerosol particle mass density of about 0.5 mg/l to about 18 mg/l, such as about 0.5 mg/l to about 12.5 mg/l, preferably of about 1.3 mg/l to about 10 mg/l, in particular of about 2 mg/l to about 9 mg/l. The pharmaceutically acceptable gas is preferably air.

The aerosol particles preferably contain less than 1 wt % impurities, in particular less than 0.5 wt % impurities. They furthermore preferably contain less than 0.5 wt % 5-MeO-DMT degradation products, in particular less than 0.2 wt % 5-MeO-DMT degradation products resulting from a chemical modification of 5-MeO-DMT as a result of a chemical reaction during aerosol formation.

In a further preferred aspect, the aerosol essentially consists of (a) air; (b) aerosol particles of 5-MeO-DMT or a pharmaceutically acceptable salt thereof.

The aerosol particles preferably contain 5-MeO-DMT in the form of the free base.

The aerosol is preferably characterized by a mass median aerodynamic diameter of less than 3 μm and more than 0.1 μm, in particular by a mass median aerodynamic diameter of less than 2 μm and more than 0.1 μm.

The aerosol may be formed by a) exposing a thin layer of 5-MeO-DMT or a pharmaceutically acceptable salt thereof, configured on a solid support, to thermal energy, and b) passing air over the thin layer of 5-MeO-DMT to produce aerosol particles. The thin layer may have a thickness of less than about 10 μm, in particular less than about 7.5 μm. It may have a thickness in the range of about 0.1 μm to about 10 μm, in particular in the range of about 0.3 μm to about 7.5 μm.

The thin layer of 5-MeO-DMT, configured on a solid support, may be exposed to thermal energy via the air passing over the thin layer. Alternatively, the thin layer of 5-MeO-DMT, configured on a solid support, may be exposed to thermal energy via the solid support.

The air passing over the thin layer may have a temperature in the range of about 180° C. to about 260° C. The air passing over the thin layer may in particular have a temperature of about 210° C. and pass over the thin layer at a rate of about 12 l/min for a duration of about 15 seconds.

The aerosol particles may be contained in a volume of equal or less than about 3 liters, in particular in a volume of about 1 to about 3 liters, such as about 2 to about 3 liters. It is preferably delivered to a patient via a single inhalation.

5-MeO-DMT or a pharmaceutically acceptable salt thereof is provided in a form suitable for inhalation in a medical context. 5-MeO-DMT and pharmaceutically acceptable salts are provided thereof in the form of aerosols. These aerosols have a suitable aerosol particle mass density so that a therapeutically effective dose of the aerosol can be administered to a patient via a single inhalation.

Aerosols useful in the present invention can be formed using thermal energy. When using thermal energy to form an aerosol of a compound, it is very difficult to predict which conditions are suitable for safe, efficient and predictable aerosolization, in particular if the aerosol is to be used for systemic delivery of that compound to a patient via the lungs. Relevant variables in this context include a) the dose of the compound, b) the morphological state in which that compound is made available for aerosolization (e.g. in crystal form, or in form as a thin layer), c) the amount of thermal energy to which the compound is exposed (defined by temperature and duration of exposure), and d) the volume of air introduced to create the aerosol (defined by flow rate and duration of air flow).

The compositions and methods described herein are for safe, efficient and predictable systemic delivery of 5-MeO-DMT or a pharmaceutically acceptable salt thereof to a patient through inhalation. “Safe” means that the aerosol particles should contain only a very small amount of impurities and 5-MeO-DMT degradation products, “efficient” means that the dosage is aerosolized to a defined extent and preferably almost completely or completely, that the aerosol has desirable physical properties for delivery of the 5-MeO-DMT or a pharmaceutically acceptable salt thereof systemically via the lungs mainly via absorption in the pulmonary alveoli, and that the aerosol can be inhaled by the patient in a single inhalation (i.e., within one deep breath), and “predictable” means that there should be almost no or no variability in the amount of degradation products, in the extent of aerosolization, and in the physical properties of the aerosol.

A suitable aerosol can be achieved by a) providing the therapeutically effective amounts of 5-MeO-DMT as a thin layer, on a solid support, b) exposing the thin 5-MeO-DMT layer to elevated controlled temperatures for a short duration of time, and c) providing a controlled amount of air so that an aerosol is formed.

A composition for delivery of a therapeutically effective amount of 5-MeO-DMT may comprise an aerosol, wherein the aerosol is formed by a) exposing a thin layer of 5-MeO-DMT, configured on a solid support, to thermal energy, and b) passing air over the thin layer of 5-MeO-DMT; wherein said aerosol has one or more of the following features: 1) it contains aerosol particles which are characterized by a mass median aerodynamic diameter of less than 3 micron, 2) it contains aerosol particles which are characterized by less than 1% wt impurities and less than 0.5% 5-MeO-DMT degradation products, 3) it can be delivered to a patient via a single inhalation.

The generation of aerosol particles characterized by a mass median aerodynamic diameter of less than 3 microns, with less than 1% wt impurities and less than 0.5% wt 5-MeO-DMT drug degradation products, in an aerosol volume which can be delivered to a patient via a single inhalation, is achieved by defining a) the dosage amount of 5-MeO-DMT contained in the thin layer of 5-MeO-DMT, b) the thickness of the thin layer of the 5-MeO-DMT, c) the thermal energy to which the thin layer of 5-MeO-DMT is exposed (defined by temperature and duration of exposure), and d) the total amount of the air which passes over the thin layer of 5-MeO-DMT (defined by airflow rate and duration of airflow).

Preferably the thin layer of 5-MeO-DMT is exposed to thermal energy via the air passing over the thin layer, in which case that air is heated. The heated air passing over the thin layer may have a temperature in the range of about 180° C. to about 260° C. The air passing over the thin layer may in particular have a temperature of about 210° C.

Alternatively, the thin layer of 5-MeO-DMT is exposed to thermal energy via the solid support, in which case the air passing over the thin layer is not heated, but the solid support is heated. The heated solid support may have a temperature in the range of about 180° C. to about 420° C.

Preferably the 5-MeO-DMT used for formation of the thin layer, on the solid support, is highly pure, with a purity of at least 99%, preferably at least 99.5%.

Preferably the dosage amount of 5-MeO-DMT contained in the thin layer of 5-MeO-DMT, configured on the solid support, is from about 1 mg to about 25 mg, preferably from about 2 mg to about 20 mg, more preferably from about 4 mg to about 20 mg. Useful specific amounts are, e.g., about 4 mg, about 6 mg, about 8 mg, about 10 mg, about 12 mg, about 14 mg, about 16 mg, about 18 mg, and about 20 mg. Preferred specific amounts are e.g. about 6 mg, about 12 mg, and about 18 mg.

Solid supports, on which 5-MeO-DMT or a pharmaceutically acceptable salt thereof is provided, can have a variety of shapes. Examples of such shapes include, without limitation, cylinders of less than 1.0 mm in diameter, boxes of less than 1.0 mm thickness and virtually any shape permeated by small (e.g., less than 1.0 mm-sized) pores. Preferably, solid supports provide a large surface to volume ratio (e.g., greater than 100 per meter) and a large surface to mass ratio (e.g., greater than 1 cm² per gram).

A solid support of one shape can also be transformed into another shape with different properties. For example, a flat sheet of 0.25 mm thickness has a surface to volume ratio of approximately 8,000 per meter. Rolling the sheet into a hollow cylinder of 1 cm diameter produces a support that retains the high surface to mass ratio of the original sheet but has a lower surface to volume ratio (about 400 per meter).

A number of different materials are used to construct the solid supports. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yarns and carbon felts; polytetrafluoroethylene and polyethylene glycol. Combinations of materials and coated variants of materials are used as well.

Where aluminum is used as a solid support, aluminum foil is a suitable material. Examples of silica, alumina and silicon based materials include amphorous silica S-5631 (Sigma, St. Louis, Mo.), BCR171 (an alumina of defined surface area greater than 2 m²/g from Aldrich, St. Louis, Mo.) and a silicon wafer as used in the semiconductor industry. Carbon yarns and felts are available from American Kynol, Inc., New York, N.Y.

Preferably the thickness of the thin layer of the 5-MeO-DMT, configured on the solid support, is less than about 10 μm, in particular less than about 7.5 μm. It may have a thickness in the range of about 0.1 μm to about 10 μm, in particular in the range of 0.3 μm to 7.5 μm.

Preferably the total amount of the air passing over the thin layer of 5-MeO-DMT is defined by a flow rate of between about 6 liters per minute and about 40 liters per minute, preferable between about 8 liters per minute and about 16 liters per minute and the duration of airflow is chosen so that the total volume of aerosol does not exceed about 3 liters, preferably is between about 1 liter and 3 liters, such as between 2 liters and 3 liters. E.g., at an airflow rate of about 6 liters per minute, the duration of airflow should be less than about 30 seconds. A useful specific airflow rate and duration is about 12 liters per minute and about 15 seconds, leading to an aerosol volume of about 3 liters. Another useful specific airflow rate and duration is 10 liters per minute and about 15 seconds, leading to leading to an aerosol volume of about 2.5 liters. Another useful specific airflow rate and duration is 8 liters per minute and about 15 seconds, leading to leading to an aerosol volume of about 2 liters. Another useful specific airflow rate and duration is 10 liters per minute and about 12 seconds, leading to leading to an aerosol volume of about 2 liters.

The aerosol formation rate is greater than 0.1 mg/sec.

The aerosol has an aerosol particle mass density of about 0.5 mg/l to about 18 mg/l, such as of about 0.5 mg/l to about 12.5 mg/l, preferably of about 1.3 mg/l to about 10 mg/l, in particular of about 2 mg/l to about 9 mg/l.

The 5-MeO-DMT aerosol particles are characterized by a mass median aerodynamic diameter of less than 3 micron and more than 0.1 micron, preferably of less than 2.5 micron and more than 0.1 micron, most preferably of less than 2 micron and more than 0.1 micron. The 5-MeO-DMT aerosol particles are characterized by less than 1% wt impurities, preferably by less than 0.5% wt impurities.

The 5-MeO-DMT aerosol particles are characterized by less than 0.5% wt 5-MeO-DMT degradation products, preferably by less than 0.2% wt 5-MeO-DMT degradation products.

A composition for delivery of a therapeutically effective amount of 5-MeO-DMT may comprise an aerosol, wherein the aerosol is formed by a) exposing a dosage amount of 12 mg 5-MeO-DMT, configured as a thin layer of less than 5 micron thickness on a solid support, to a temperature of 210° C. via passing heated air over the thin layer for a duration of 15 seconds; wherein said aerosol has one or more of the following features: 1) it contains aerosol particles which are characterized by a mass median aerodynamic diameter of less than 3 micron, 2) it contains aerosol particles which are characterized by less than 1% impurities and less than 0.5% wt 5-MeO-DMT degradation products, 3) it can be delivered to a patient via a single inhalation.

A skilled person, knowing the aerosol characteristics and the aerosolization conditions defined in the present invention, can identify suitable vaporization devices or systems, which lead to the required aerosol characteristics. Examples of such suitable vaporization devices or systems include e.g. the Volcano Medic Vaporization System with the associated dosing capsules with drip pad (Storz & Bickel, Germany; as disclosed in e.g. EP 0 933 093 B1, and EP 1 884 254 B1 and Registered Community Design 003387299-0001) and the Staccato device (Alexza Pharmaceuticals, Mountain View, USA; as disclosed e.g. in U.S. Pat. Nos. 7,458,374 B2, 9,370,629 B2 and 9,687,487 B2). The aerosol generated may be collected in a balloon and inhaled by the patient from the balloon.

### Dosing Regimen

The present invention also provides dose ranges, particular doses as well as dosing regimens (administration schemes).

The invention is in part based on the inventor's conclusion that the occurrence of a peak psychedelic experience during the acute phase after administration of 5-MeO-DMT is driving its therapeutic benefit in patients suffering from PPD, in particular one or more of the aspects defined above, either in a causal relationship or at least as a surrogate behavioural marker for the underlying unknown therapeutic mechanism.

Consequently, achieving peak experiences more rapidly, in a larger proportion of patients and with better reproducibility in an individual patient, compared with previously tested psychedelic agents and dosing regimens, will lead to a better therapeutic profile.

Further, the present invention also relies on the short duration of action of 5-MeO-DMT and the absence of relevant tolerance (i.e., the absence of diminished or no psychedelic effects after re-administration), as a basis for enabling a dosing regimen with frequent re-administrations (such as more than once daily, or daily), which are designed to increase the rate of occurrence of peak experiences, thereby increasing the therapeutic benefit. Such repeat administrations within short time also allow an intraindividual dose-optimization which reduces the risk of overdosing, which may otherwise lead to somatic side effects, such as the serotonin syndrome, negative psychic reactions, such as flash-backs of the experience at later timepoints, induction of mania or hypomania or to less meaningful psychedelic experiences with few or no memories of the altered state (so-called “white-outs”). Further, starting with a low dose allows familiarization of the patient with the psychedelic experience in general, and allows preparation for the more intense symptoms to occur at the higher doses, which will positively influence the experience at those higher doses. Also, the prospect of being able to initiate treatment with a low dose will increase patient acceptance of the therapeutic approach and improve overall compliance rates on the patient population level.

Frequent re-administrations of a serotonergic psychedelic with the aim to increase the rate and tailor the reproducibility of peak experiences and to improve the therapeutic effect, reduce the side effects and improve the compliance rates may not be possible with other psychedelics, due to the late onset and long duration of psychedelic effects and due to the rapid development of tolerance (i.e. diminished or no psychedelic effects after re-administration) which can last for several days.

A patient as defined herein, diagnosed with postpartum depression, including a treatment-resistant form of this disorder, and including this disorder associated with suicidal ideation, is treated by administration of 5-MeO-DMT. In a preferred embodiment, the 5-MeO-DMT is administered as a monotherapy, i.e., the patient does not receive any other treatment for PPD or symptoms associated with PPD.

The dosage amount of 5-MeO-DMT administered to a patient, as defined herein, diagnosed with postpartum depression, including a treatment-resistant form of this disorder, and including this disorder associated with suicidal ideation, is in the range of about 1 mg to about 25 mg, or any amount of range therein, preferably from about 2 mg to about 20 mg, more preferably from about 4 mg to about 20 mg. Useful specific amounts are e.g. about 4 mg, about 6 mg, about 8 mg, about 10 mg, about 12 mg, about 14 mg, about 16 mg, about 18 mg, and about 20 mg. Patients may also be treated with an equimolar dose of a pharmaceutically acceptable salt of 5-MeO-DMT, such as the hydrobromide salt. Note that in this specification, when ranges are set forth, such as “about 1 mg to about 25 mg,” the inventor contemplates all discrete values within that range, some of which are specifically mentioned, but all of which are not—simply for the purpose of brevity.

In preferred embodiments the improved methods for the treatment of a patient, as defined herein, diagnosed with postpartum depression, including a treatment-resistant form of this disorder, and including this disorder associated with suicidal ideation, with a therapeutically effective amount of 5-MeO-DMT, comprise the occurrence of a clinical response not later than about 2 hours after administration of 5-MeO-DMT.

In preferred embodiments the improved methods for the treatment of a patient, as defined herein, diagnosed with postpartum depression, including a treatment-resistant form of this disorder, and including this disorder associated with suicidal ideation, with a therapeutically effective amount of 5-MeO-DMT, comprise the persistence of a clinical response, including a clinical response which occurred not later than about 2 hours after administration of 5-MeO-DMT, until at least about 6 days after the last administration of 5-MeO-DMT, preferably until at least about 14 days after the last administration of 5-MeO-DMT, more preferably until at least about 28 days after the last administration of 5-MeO-DMT.

In preferred embodiments the improved methods for the treatment of a patient, as defined herein, diagnosed with postpartum depression, including a treatment-resistant form of this disorder, and including this disorder associated with suicidal ideation, with a therapeutically effective amount of 5-MeO-DMT comprise the administration of more than a single dose of 5-MeO-DMT.

In a preferred embodiment this more than a single dose of 5-MeO-DMT is administered to a patient in one or more treatment blocks, each block consisting of 2 to 7 administrations, with not less than about 1 hour and not more than about 24 hours between each administration within each treatment block, and not less than about 6 days between the end of one treatment block and the start of the next treatment block.

In an even more preferred embodiment this more than a single dose of 5-MeO-DMT is administered to a patient in one or more treatment blocks, each block consisting of 1 to 3 administrations, with about 24 hours between each administration within each treatment block, and not less than about 6 days between the end of one treatment block and the start of the next treatment block.

In a most preferred embodiment this more than a single dose of 5-MeO-DMT is administered to a patient in one or more treatment blocks, each block consisting of 1 to 3 administrations, with about 1 to 4 hours, preferably 1 to 2 hours, between each administration within each treatment block, and not less than about 6 days between the end of one treatment block and the start of the next treatment block.

In an embodiment the dosage amount of the 5-MeO-DMT administered to an individual patient in each of the administrations and in each of the treatment blocks is constant for that individual patient and is selected from about 1 mg to about 25 mg, preferably from about 2 mg to about 20 mg, more preferably from about 4 mg to about 20 mg. Useful specific amounts are e.g. about 4 mg, about 6 mg, about 8 mg, about 10 mg, about 12 mg, about 14 mg, about 16 mg, about 18 mg, and about 20 mg.

In a preferred embodiment the dosage amount of the 5-MeO-DMT administered to an individual patient is selected from about 2 mg to about 8 mg for the first administration within each treatment block, and then increases with each subsequent administration within each treatment block until the earlier of 20 mg being reached or all administrations within that treatment block being administered.

In an even more preferred embodiment the dosage amount of the 5-MeO-DMT administered to an individual patient is selected from about 2 mg to about 8 mg for the first administration within each treatment block, and then increases with each subsequent administration within each treatment block until the earlier of 20 mg being reached or all administrations within that treatment block being administered or the patient having experienced a peak psychedelic experience or the supervising physician having decided that further dose increases are inappropriate based on observed side effects.

For embodiments where the dosage amount increases for subsequent administrations, the dosage amount for the next administration is determined by adding about 2 mg to about 10 mg, preferably about 4 mg to about 8 mg, most preferably about 6 mg, to the dosage amount of the prior administration. For example, if the dosage amount of the first administration was 6 mg and the dosage amount increase is 6 mg, unless one of the previously mentioned stopping criteria has been reached, then the dosage amount of the second administration will be 12 mg. Preferably, the dosage amount for the third administration will be 18 mg.

In a preferred embodiment the dosage amount of the 5-MeO-DMT administered to an individual patient in each treatment block is selected from about 2 mg to about 8 mg for the first administration, and then increased, unless the patient has already experienced a peak psychedelic experience within that treatment block or the supervising physician has decided that further dose increases are inappropriate based on observed side effects, to a dosage selected from about 8 mg to about 14 mg for the second administration, and from about 14 mg to about 20 mg for the third administration. Useful specific amounts for the first, second and third administration are e.g. about 6 mg, about 12 mg, and about 18 mg.

In a further preferred embodiment the dosage amount of the 5-MeO-DMT administered to an individual patient is selected from about 2 mg to about 8 mg for the first administration of the first treatment block, and then increases with each subsequent administration within that first treatment block until the earlier of 20 mg being reached or all administrations within that treatment block being administered or the patient having experienced a peak psychedelic experience or the supervising physician having decided that further dose increases are inappropriate based on observed side effects, with that highest dosage in that first treatment block being used as the dosage for all subsequent treatment blocks and administrations within those subsequent treatment blocks. For example, if the highest dosage in the first treatment block was 18 mg because the patient experienced a peak psychedelic experience at that dose, then the dosage for all subsequent treatment blocks and administrations within those subsequent treatment blocks will be 18 mg.

In a most preferred embodiment the dosage amount of the 5-MeO-DMT administered to an individual patient is selected from about 2 mg to about 8 mg for the first administration of the first treatment block, and then increased, unless the patient has already experienced a peak psychedelic experience within that treatment block or the supervising physician has decided that further dose increases are inappropriate based on observed side effects, to a dosage selected from about 8 mg to about 14 mg for the second administration of the first treatment block, and from about 14 mg to about 20 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 6 mg, about 12 mg, and about 18 mg.

It is understood that a pharmaceutically acceptable salt of 5-MeO-DMT can also be used in all of the above dosing regimen, and that the appropriate weight amounts of a salt to be administered can be calculated from the stated weight amounts of the free base, assuming that equimolar amounts are used.

According to the invention, 5-MeO-DMT is preferably not administered together with a MAO inhibitor.

The occurrence of a “peak psychedelic experience” in a patient can be identified through achievement of at least 60% of the maximum possible score in each of the four subscales (mystical, positive mood, transcendence of time and space, and ineffability) of the 30-item revised Mystical Experience Questionnaire (MEQ-30) (as described in Barrett F S, J Psychopharmacol. 2015; 29(11):1182-90).

The occurrence of a “peak psychedelic experience” in a patient can also be identified through achievement of at least 60% of the maximum possible score of the Oceanic Boundlessness (OBN) dimension of the Altered States of Consciousness (ASC) questionnaire (as described in Roseman L et al., Front Pharmacol. 2018; 8:974).

In accordance with the invention, the occurrence of a “peak psychedelic experience” in a patient is preferably identified through achievement of a score of at least 75 in the Peak Experience Scale (PES) Total Score, also referred to as the Peak Psychedelic Experience Questionnaire (PPEQ), which averages answers scored by the patient from 0 to 100 for the following three questions: 1. How intense was the experience; 2. To what extent did you lose control; 3. How profound (i.e. deep and significant) was the experience?

### Further Aspects of the Invention

1. 5-Methoxy-N,N-dimethyltryptamine (5-MeO-DMT) or a pharmaceutically acceptable salt thereof for use in treating a patient suffering from postpartum depression (PPD).

2. 5-MeO-DMT or a pharmaceutically acceptable salt thereof for use as in aspect 1, wherein the patient has a Montgomery-Åsberg Depression Rating Scale (MADRS) score of 20 or more or a 17-item Hamilton Depression Rating Scale (HAM-D) score of 16 or more.

3. 5-MeO-DMT or a pharmaceutically acceptable salt thereof for use as in aspect 2, wherein the patient has a MADRS score of 28 or more or a HAM-D score of 22 or more.

4. 5-MeO-DMT or a pharmaceutically acceptable salt thereof for use as in aspect 2, wherein the patient has a MADRS score of 35 or more or by a HAM-D score of 27 or more.

5. 5-MeO-DMT or a pharmaceutically acceptable salt thereof for use as in aspects 1 to 4, wherein the patient is diagnosed with a treatment-resistant form of postpartum depression.

6. 5-MeO-DMT or a pharmaceutically acceptable salt thereof for use as in aspects 1 to 5, wherein the patient suffers in addition from suicidal ideation.

7. 5-MeO-DMT or a pharmaceutically acceptable salt thereof for use as in aspects 1 to 6, wherein the patient suffers in addition from slightly compromised, compromised or severely compromised maternal functioning.

8. 5-MeO-DMT or a pharmaceutically acceptable salt thereof for use as in aspect 7, wherein the patient has a Barkin Index of Maternal Functioning (BIMF) score of 95 or below such as 80 or below, in particular 65 or below.

9. 5-MeO-DMT or a pharmaceutically acceptable salt thereof for use as in aspects 1 to 8, wherein the 5-MeO-DMT or salt thereof is administered at a dose or in a dosage regimen that causes the patient to experience a peak psychedelic experience.

10. 5-MeO-DMT or a pharmaceutically acceptable salt thereof for use as in aspects 1 to 9, wherein a dosage of about 4 mg to about 20 mg 5-MeO-DMT is administered, or wherein equimolar amounts of the pharmaceutically acceptable salt are administered instead of 5-MeO-DMT.

11. 5-MeO-DMT or a pharmaceutically acceptable salt thereof for use as in aspects 1 to 9, wherein a dosage of about 6 mg; or of about 12 mg; or of about 18 mg is administered, or wherein equimolar amounts of the pharmaceutically acceptable salt are administered instead of 5-MeO-DMT.

12. 5-MeO-DMT or a pharmaceutically acceptable salt thereof for use as in aspects 1 to 10, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

13. 5-MeO-DMT or a pharmaceutically acceptable salt thereof for use as in aspects 1 to 12, wherein the 5-MeO-DMT is administered in a dosage from about 2 mg to about 8 mg for a first administration, and then increased, unless the patient has already experienced a peak psychedelic experience, to a dosage from about 8 mg to about 14 mg for a second administration, and then increased, unless the patient has already experienced a peak psychedelic experience, to a dosage from about 14 mg to about 20 mg for a third administration, or wherein equimolar amounts of the pharmaceutically acceptable salt are administered instead of 5-MeO-DMT.

14. 5-MeO-DMT or a pharmaceutically acceptable salt thereof for use as in aspect 13, wherein the first dosage of 5-MeO-DMT is about 6 mg, the second dosage of 5-MeO-DMT is about 12 mg, and the third dosage of 5-MeO-DMT is about 18 mg; or wherein equimolar amounts of the pharmaceutically acceptable salt are administered instead of 5-MeO-DMT.

15. 5-MeO-DMT or a pharmaceutically acceptable salt thereof for use as in aspects 12 to 14, wherein the interval between two administrations is not less than 1 hour and not more than 24 hours, such as about 1 to 4 hours, preferably about 1 to 2 hours.

16. 5-MeO-DMT or a pharmaceutically acceptable salt thereof for use as in aspects 9 to 15, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

17. 5-MeO-DMT or a pharmaceutically acceptable salt thereof for use as in aspect 16, wherein the occurrence of a peak psychedelic experience is identified through achievement of a Peak Experience Scale (PES) Total Score of at least 75.

18. 5-MeO-DMT or a pharmaceutically acceptable salt thereof for use as in aspects 1 to 17, wherein the 5-MeO-DMT or a pharmaceutically acceptable salt thereof is administered via inhalation or by nasal, buccal or sublingual administration.

19. 5-MeO-DMT or a pharmaceutically acceptable salt thereof for use as in aspect 18, wherein 5-MeO-DMT or a pharmaceutically acceptable salt thereof is administered in the form of an aerosol comprising (a) a pharmaceutically acceptable gas; (b) aerosol particles of 5-methoxy-N,N-dimethyltryptamine (5-MeO-DMT) or a pharmaceutically acceptable salt thereof, wherein the aerosol has an aerosol particle mass density of about 0.5 mg/l to about 18 mg/l, such as to about 12.5 mg/l.

20. 5-MeO-DMT or a pharmaceutically acceptable salt thereof for use as in aspect 19, wherein the aerosol is generated by a) exposing a thin layer of 5-MeO-DMT or a pharmaceutically acceptable salt thereof, configured on a solid support, to thermal energy, and b) passing air over the thin layer to produce aerosol particles.

21. 5-MeO-DMT or a pharmaceutically acceptable salt thereof for use as in aspects 18 to 20, wherein the dosage amount of 5-MeO-DMT or a pharmaceutically acceptable salt to be administered to the patient is inhaled with a single breath.

22. 5-MeO-DMT for use as in aspects 18 to 21, wherein the 5-MeO-DMT is used in the form of the free base.

23. 5-MeO-DMT or a pharmaceutically acceptable salt thereof for use as in aspects 1 to 22, wherein a clinical response, as reflected by a reduction in the Clinical Global Impression-Severity (CGI-S) score, occurs not later than about 2 hours after the last administration of 5-MeO-DMT or a pharmaceutically acceptable salt thereof.

24. 5-MeO-DMT or a pharmaceutically acceptable salt thereof for use as in aspects 1 to 23, wherein a clinical response, as reflected by a reduction in the Clinical Global Impression-Severity (CGI-S) score, occurs on day 1, for instance, about 24 hours after the last administration of 5-MeO-DMT or a pharmaceutically acceptable salt thereof.

25. 5-MeO-DMT or a pharmaceutically acceptable salt thereof for use as in aspects 1 to 24, wherein a clinical response, as reflected by a reduction in the Clinical Global Impression-Severity (CGI-S) score, persists until at least 6 days after the last administration of 5-MeO-DMT or a pharmaceutically acceptable salt thereof.

26. 5-MeO-DMT or a pharmaceutically acceptable salt thereof for use as in aspects 1 to 25, wherein a clinical response, as reflected by a reduction in the Clinical Global Impression-Severity (CGI-S) score, persists until at least 14 days after the last administration of 5-MeO-DMT or a pharmaceutically acceptable salt thereof.

27. 5-MeO-DMT or a pharmaceutically acceptable salt thereof for use as in aspects 1 to 26, wherein a clinical response, as reflected by a reduction in the Clinical Global Impression-Severity (CGI-S) score, persists until at least 28 days after the last administration of 5-MeO-DMT or a pharmaceutically acceptable salt thereof.

28. 5-MeO-DMT or a pharmaceutically acceptable salt thereof for use as in aspects 1 to 27, wherein a clinical response, as assessed by at least 50% improvement of the MADRS or HAM-D score, compared to the respective score prior to treatment, occurs not later than about 2 hours after the last administration of 5-MeO-DMT or a pharmaceutically acceptable salt thereof.

29. 5-MeO-DMT or a pharmaceutically acceptable salt thereof for use as in aspects 1 to 28, wherein a clinical response, as assessed by at least 75% improvement of the MADRS or HAM-D score, compared to the respective score prior to treatment, occurs not later than about 2 hours after the last administration of 5-MeO-DMT or a pharmaceutically acceptable salt thereof.

30. 5-MeO-DMT or a pharmaceutically acceptable salt thereof for use as in aspects 1 to 29, wherein a remission of depressive symptoms, as assessed by a MADRS score equal to or less than 10, or a HAM-D score equal to or less than 7, occurs not later than about 2 hours after the last administration of 5-MeO-DMT or a pharmaceutically acceptable salt thereof.

31. 5-MeO-DMT or a pharmaceutically acceptable salt thereof for use as in aspects 1 to 30, wherein a remission of depressive symptoms, as assessed by a MADRS score equal to or less than 10, or a HAM-D score equal to or less than 7, occurs on day 1, for instance, about 24 hours after the last administration of 5-MeO-DMT or a pharmaceutically acceptable salt thereof.

32. 5-MeO-DMT or a pharmaceutically acceptable salt thereof for use as in aspects 1 to 31, wherein the clinical response, as assessed by at least 50% improvement of the MADRS or HAM-D score, compared to the respective score prior to treatment, persists until at least 6 days after the last administration of 5-MeO-DMT or a pharmaceutically acceptable salt thereof.

33. 5-MeO-DMT or a pharmaceutically acceptable salt thereof for use as in aspects 1 to 32, wherein there is a clinical response, as assessed by at least 75% improvement of the MADRS or HAM-D score, compared to the respective score prior to treatment, on day 7 after the last administration of 5-MeO-DMT or a pharmaceutically acceptable salt thereof.

34. 5-MeO-DMT or a pharmaceutically acceptable salt thereof for use as in aspects 1 to 33, wherein the patient is in remission of depressive symptoms, as assessed by a MADRS score equal to or less than 10, or a HAM-D score equal to or less than 7, on day 7 after the last administration of 5-MeO-DMT or a pharmaceutically acceptable salt thereof.

35. 5-MeO-DMT or a pharmaceutically acceptable salt thereof for use as in aspects 1 to 34, wherein the clinical response, as assessed by at least 50% improvement of the MADRS or HAM-D score, compared to the respective score prior to treatment, persists until at least 14 days after the last administration of 5-MeO-DMT or a pharmaceutically acceptable salt thereof.

36. 5-MeO-DMT or a pharmaceutically acceptable salt thereof for use as in aspects 1 to 35, wherein there is a clinical response, as assessed by at least 75% improvement of the MADRS or HAM-D score, compared to the respective score prior to treatment, on day 14 after the last administration of 5-MeO-DMT or a pharmaceutically acceptable salt thereof.

37. 5-MeO-DMT or a pharmaceutically acceptable salt thereof for use as in aspects 1 to 36, wherein the patient is in remission of depressive symptoms, as assessed by a MADRS score equal to or less than 10, or a HAM-D score equal to or less than 7, on day 14 after the last administration of 5-MeO-DMT or a pharmaceutically acceptable salt thereof.

38. 5-MeO-DMT or a pharmaceutically acceptable salt thereof for use as in aspects 1 to 37, wherein the clinical response, as assessed by at least 50% improvement of the MADRS or HAM-D score, compared to the respective score prior to treatment, persists until at least 28 days after the last administration of 5-MeO-DMT or a pharmaceutically acceptable salt thereof.

39. 5-MeO-DMT or a pharmaceutically acceptable salt thereof for use as in aspects 1 to 38, wherein there is a clinical response, as assessed by at least 75% improvement of the MADRS or HAM-D score, compared to the respective score prior to treatment, on day 28 after the last administration of 5-MeO-DMT or a pharmaceutically acceptable salt thereof.

40. 5-MeO-DMT or a pharmaceutically acceptable salt thereof for use as in aspects 1 to 39, wherein the patient is in remission of depressive symptoms, as assessed by a MADRS score equal to or less than 10, or a HAM-D score equal to or less than 7, on day 28 after the last administration of 5-MeO-DMT or a pharmaceutically acceptable salt thereof.

41. 5-Methoxy-N,N-dimethyltryptamine (5-MeO-DMT) or a pharmaceutically acceptable salt thereof for use in any of aspects 1 to 40, wherein the patient is a breastfeeding mother who is advised to discontinue breastfeeding until 48 hours after the last administration of 5-MeO-DMT or a pharmaceutically acceptable salt thereof.

42. 5-Methoxy-N,N-dimethyltryptamine (5-MeO-DMT) or a pharmaceutically acceptable salt thereof for use in any of aspects 1 to 40, wherein the patient is a breastfeeding mother who is advised to discontinue breastfeeding until 24 hours after the last administration of 5-MeO-DMT or a pharmaceutically acceptable salt thereof.

43. 5-Methoxy-N,N-dimethyltryptamine (5-MeO-DMT) or a pharmaceutically acceptable salt thereof for use in any of aspects 1 to 40, wherein the patient is a breastfeeding mother who is advised to discontinue breastfeeding until 6 hours after the last administration of 5-MeO-DMT or a pharmaceutically acceptable salt thereof.

44. 5-Methoxy-N,N-dimethyltryptamine (5-MeO-DMT) or a pharmaceutically acceptable salt thereof for use in any of aspects 1 to 40, wherein the patient is a breastfeeding mother who is advised to discontinue breastfeeding until 3 hours after the last administration of 5-MeO-DMT or a pharmaceutically acceptable salt thereof.

45. 5-Methoxy-N,N-dimethyltryptamine (5-MeO-DMT) or a pharmaceutically acceptable salt thereof for use in any of aspects 1 to 40, wherein the patient is a breastfeeding mother who is advised to discontinue breastfeeding until 2 hours after the last administration of 5-MeO-DMT or a pharmaceutically acceptable salt thereof.

46. 5-MeO-DMT or a pharmaceutically acceptable salt thereof for use as in aspects 1 to 45, wherein the treatment improves maternal functioning.

47. 5-MeO-DMT or a pharmaceutically acceptable salt thereof for use as in aspect 46, wherein the improvement relates to one or more, in particular two or more functional domains according to the Barkin Index of Maternal Functioning (BIMF) selected from self-care, infant care, mother-child interaction, psychological wellbeing of the mother, social support, management, and adjustment.

48. 5-MeO-DMT or a pharmaceutically acceptable salt thereof for use as in aspect 46 or 47, wherein the BIMF score is improved by 10% or more, preferably by 20% or more.

### EXAMPLES

The following Examples are listed to aid understanding of the invention and are not intended and should not be construed to limit in any way the invention set forth in the claims which follow thereafter.

### Example 1-5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the volunteer or patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.

Step 2: 200 μl of the solution is transferred to a dosing capsule containing the drip pad (Storz & Bickel, Germany), and then the dosing capsule is closed with its lid.

Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 l/min. The heated air will flow through the dosing capsule, allowing the ethanol to evaporate, with the target dosage of 5-MeO-DMT being left in the capsule, as a thin layer covering the stainless-steel wire mesh. Accurate preparation of the dosing capsule can be confirmed by demonstrating that the final weight increase of the capsule compared to the weight of the empty capsule is about equal to the target dosage of 5-MeO-DMT.

Step 4: The prepared dosing capsule is removed from the filling chamber. It is then transferred to the filling chamber of a second Volcano Medic Vaporizer, which has been pre-heated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 l/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.

Step 5: The balloon is then disconnected from the filling chamber, which automatically closes the valve. After attachment of the mouthpiece to the balloon, the aerosol is ready for immediate administration to the volunteer or patient.

Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

Further details regarding the administration of 5-MeO-DMT by inhalation are disclosed in Example 1 of WO 2020/169850 A1, the contents of which is incorporated herein by reference.

### Example 2—Preparation of 5-MeO-DMT in High Purity

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC as described in WO 2020/169850 A1. The purity was found to be 99.74% area.

The results from the analysis further indicate that the level of individual impurities was below 0.10% area. Solvent analysis of sample indicated an MTBE level of 17 ppm.

### Example 3—Preparation of 5-MeO-DMT Hydrobromide Salt

5-MeO-DMT HBr was prepared on a 100 mg scale.

5-MeO-DMT free base was combined with isopropyl acetate (10 vols), and the resulting solution of 5-MeO-DMT was heated to 50° C. HBr was charged (1 M in ethanol, 1 eq) in one single aliquot. The mixture was held at temperature and equilibrated for 3 hours.

After 1 hour, a suspension had formed. The suspension was finally cooled to room temperature and equilibrated for 18 hours. Solids were isolated by filtration and dried in vacuo at 40° C. for 18 hours.

An off-white crystalline material was obtained.

The salt has a melting point of 174° C. and is characterized by an X-ray diffraction pattern comprising peaks at 14.5°2•±0.2°2•; 16.7°2•±0.2°2•; 17.0°2•±0.2°2•; 20.6°2•±0.2°2•; 20.7°2•±0.2°2•; 21.4°2•±0.2°2•; 24.2°2•±0.2°2•; 24.8°2•±0.2°2•; 25.3°2•±0.2°2•; 27.4°2•±0.2°2•; measured using Cu K radiation.

### Example 4—Determination of Inhibition Constants for Central 5-HT1A and 5-HT2A Receptors in Post-Mortem Human Brain Membrane Preparations

In this study, the affinity of three psychedelic test compounds (psilocin, DMT and 5-MeO-DMT) for 5-HT1A and 5-HT2A receptors in post-mortem human brain tissue from the hippocampus and frontal cortex, respectively, was determined using the technique of radioligand binding.

Human brain samples were obtained from the Edinburgh Sudden Death Brain Bank. All donors were sudden deaths with no prior history of coma, psychiatric or neurological disorders and under the age of 65 with a post-mortem interval of less than or equal to 72 hours.

### Binding to 5-HT1A Receptors in Post-Mortem Human Hippocampus

Hippocampus was homogenised in ice-cold 0.25 M sucrose (1:30 w/v) using a motor driven Teflon pestle (12 strokes at 120 rpm). Myelin and cell debris were removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold membrane preparation buffer, (1:100 w/v) using a tight-fitting glass/Teflon homogeniser (12 strokes, 800 rpm) and centrifuged at 20,500 g for 10 minutes. The pellet was resuspended in ice-cold membrane preparation buffer and incubated at 37° C. for 10 minutes before being centrifuged at 20,500 g for 10 minutes. The pellet was resuspended and centrifuged a final time to wash the tissue (20,500×g for 10 mins). The resulting pellet was then resuspended in ice-cold assay buffer at a tissue concentration equivalent to 3.125 mg wet weight of tissue/ml. All centrifugations were carried out at 4° C. The membrane preparation buffer consisted of 50 mM Tris-HCl, pH 7.7, 4 mM CaCl₂ and 0.1% ascorbic acid. The assay buffer consisted of 50 mM Tris, pH 7.7, 4 mM CaCl₂, 0.1% ascorbic acid and 10 μM Pargyline.

For saturation binding analysis, hippocampal membranes (400 μl; equivalent 1.25 mg wet weight tissue/tube) was incubated with 50 μl of 0.075-9.6 nM [³H]8-OH-DPAT and either 50 μl of assay buffer (total binding) or 50 μl of 1 μM WAY 100635 (non-specific binding) at 25° C. for 30 minutes. The wash buffer consisted of 50 mM Tris, pH 7.7.

In a displacement assay, hippocampal membranes (400 μl; equivalent 1.25 mg wet weight tissue/tube) were incubated with 50 μl of 0.6 nM [³H]8-OH-DPAT and either 50 μl of assay buffer (total binding) or 50 μl of 1 •M WAY 100635 (non-specific binding) or 50 μl of one of the test compounds in one of ten concentrations between 1 and 10000 nM at 25° C. for 30 minutes.

Membrane bound radioactivity was recovered by filtration under vacuum through Skatron 11731 filters, pre-soaked in 0.5% polyethylenimine (PEI) using a Skatron cell harvester. Filters were rapidly washed with ice-cold wash buffer (wash setting 0,9,9) and radioactivity determined by liquid scintillation counting (1 ml Packard MV Gold scintillator).

The concentration of compound required to inhibit 50% of specific binding (IC₅₀) and the Hill Slope were calculated by using non-linear regression. The Kᵢ was calculated using the one-site binding model allowing for ligand depletion.

### Binding to 5-HT2A Receptors in Post-Mortem Human Frontal Cortex

Frontal cortex was homogenised in ice-cold 0.25 M sucrose (1:30 w/v) using a motor driven Teflon pestle (12 strokes at 120 rpm). Myelin and cell debris was removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold 50 mM Tris-HCl assay buffer, pH 7.4 (1:100 w/v), homogenised using a tight-fitting glass/Teflon homogeniser (12 strokes, 800 rpm) and centrifuged at 20,500 g for 10 minutes. The pellet was centrifuged a further two times to wash the tissue (20,500×g for 10 mins). The resulting pellet was then resuspended in ice-cold 50 mM Tris-HCl assay buffer, pH 7.4 at a tissue concentration equivalent to 10 mg wet weight of tissue/ml. All centrifugations were carried out at 4° C.

For saturation binding analysis, frontal cortical membranes (400 μl; equivalent to 4 mg wet weight of tissue/tube) were incubated with 50 μl of 0.00625-0.8 nM [³H]MDL-100,907 and either 50 μl of assay buffer or 50 μl of 10 •M ketanserin (non-specific binding) at 25° C. for 60 minutes. The assay and wash buffer consisted of 50 mM Tris-HCl buffer pH 7.4.

In a displacement assay, frontal cortical membranes (400 μl; equivalent 4 mg wet weight tissue/tube) was incubated with 50 μl of 0.1 nM [3H]MDL-100,907 and either 50 μl of assay buffer (total binding) or 50 μl of 10 M ketanserin (non-specific binding) or 50 μl of one of the test compounds in one of ten concentrations between 1 and 10000 nM at 25° C. for 60 minutes.

Membrane bound radioactivity was recovered and determined as above. Data analysis was also as above.

### Results

The dissociation constant (K_{d} value) of [³H]8-OH-DPAT for 5-HT1A receptors in hippocampal membranes from post-mortem human brain tissue was determined for each of the three donors. The dissociation constants (K_{d} values) obtained were 0.51, 0.28 and 0.52 nM, respectively.

Mean inhibition constants (Kᵢ values) for psilocin, DMT and 5-MeO-DMT were 48, 38 and 1.80 nM (mean n=3), respectively. All compounds gave Hill slopes approximating to unity, suggesting a one-site binding model.

The dissociation constant (K_{d} values) of [³H]MDL-100,907 for 5-HT2A receptors in frontal cortical membranes from post-mortem human brain tissue was determined for each of the three donors. The dissociation constants (K_{d} values) obtained were 0.11, 0.08 and 0.08 nM, respectively.

Mean inhibition constants (Kᵢ values) for psilocin, DMT and 5-MeO-DMT were 37, 117 and 122 nM (mean n=3), respectively. All compounds gave Hill slopes approximating to unity, suggesting a one-site binding model.

The selectivity ratio of psilocin, DMT and 5-MeO-DMT for 5-HT2A over 5-HT1A receptors was 0.78, 3.1 and 68, respectively.

### Example 5—Clinical Trial in Patients Suffering from TRD

A Phase 1/2 clinical trial of 5-MeO-DMT, administered via inhalation as described herein, in patients with treatment-resistant major depressive disorder (TRD) has been completed. This trial was designed in two parts. Part A was an open-label, single-arm, single-dose Phase 1 trial with two dose levels (12 mg (n=4) and 18 mg (n=4)). Part B was an open-label, single-arm Phase 2 trial applying an individualized dosing regimen with intra-patient dose escalation with 5-MeO-DMT. Patients (n=8) received at least one and up to three doses of 5-MeO-DMT (6 mg, 12 mg and 18 mg) in a single day, with higher doses only being administered if a peak experience was not achieved at the previously administered dose. The primary endpoint of Part A was to assess the safety and tolerability of single dosing of 5-MeO-DMT in patients with TRD. The primary endpoint of Part B was to assess the effects on the severity of depression, as assessed by the proportion of patients in remission on day seven after dosing, defined as a MADRS total score of less than or equal to 10.

In Part A, 3 of 4 patients in both groups (12 mg and 18 mg) experienced at least one ADR, all of which were mild and resolved spontaneously. No SAEs were reported.

Two of four patients (50%) in the 12 mg group and one of four patients (25%) in the 18 mg group had a MADRS remission on day seven after dosing, and one further patient (25%) in the 18 mg group had a MADRS clinical response on day seven after dosing. The mean MADRS change from baseline at day seven was −21.0 (−65%) in the 12 mg group and −12.8 (−41%) in the 18 mg group.

In Part B, 7 of 8 patients (87.5%) experienced at least one ADR. All ADRs resolved spontaneously. No SAEs were reported.

The primary endpoint was met with seven of eight patients (87.5%) achieving a MADRS remission on day seven (p<0.0001). The mean MADRS change from baseline at day seven was 24.4 (76%).

No clinically significant changes were observed in either Part A or Part B in any of the safety laboratory analyses, vital signs, psychiatric safety assessments or measures of cognitive function.

Results are summarized in the tables below.

TABLE 2-A
Scores recorded against relevant MADRS and BPRS items for patients assigned to
intra-day individualised dosing regimen (IDR). The item scores represent the sum
of the individual patient scores for all patients (n = 8) in the IDR group.
Assessment 2 hours (MADRS) or 3 hours (BPRS) after administration of the last dose.

| | | Baseline | 2/3 | Improvement | Percentage |
|---|---|---|---|---|---|
| Scale item | Scale | IDR | hours | 2/3 hours | improvement |
| Concentration Difficulties | MADRS | 30 | 11 | 19 | 63.33% |
| Lassitude | MADRS | 27 | 10 | 17 | 62.96% |
| Inability to Feel | MADRS | 36 | 12 | 24 | 66.67% |
| Inner Tension | MADRS | 26 | 11 | 15 | 57.69% |
| Pessimistic Thoughts | MADRS | 28 | 7 | 21 | 75.00% |
| Suicidal Thoughts | MADRS | 11 | 3 | 8 | 72.72% |
| Guilt Feelings | BPRS | 34 | 14 | 20 | 58.82% |
| Tension | BPRS | 16 | 11 | 5 | 31.25% |
| Anxiety | BPRS | 37 | 19 | 18 | 48.65% |
| Emotional Withdrawal | BPRS | 13 | 8 | 5 | 38.46% |
| Blunted Affect | BPRS | 15 | 11 | 4 | 26.67% |

TABLE 2-B
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 1.

| | | Baseline | | Improvement | Percentage |
|---|---|---|---|---|---|
| Scale item | Scale | IDR | Day 1 | at Day 1 | improvement |
| Reduced Sleep | MADRS | 25 | 12 | 13 | 52.00% |
| Concentration Difficulties | MADRS | 30 | 1 | 29 | 96.67% |
| Lassitude | MADRS | 27 | 5 | 22 | 81.48% |
| Inability to Feel | MADRS | 36 | 2 | 34 | 94.44% |
| Inner Tension | MADRS | 26 | 6 | 20 | 76.92% |
| Pessimistic Thoughts | MADRS | 28 | 4 | 24 | 85.71% |
| Suicidal Thoughts | MADRS | 11 | 1 | 10 | 90.91% |
| Guilt Feelings | BPRS | 34 | 11 | 23 | 67.65% |
| Tension | BPRS | 16 | 11 | 5 | 31.25% |
| Anxiety | BPRS | 37 | 16 | 21 | 56.76% |
| Emotional Withdrawal | BPRS | 13 | 8 | 5 | 38.46% |
| Blunted Affect | BPRS | 15 | 8 | 7 | 46.67% |

TABLE 2-C
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 7.

| | | Baseline | | Improvement | Percentage |
|---|---|---|---|---|---|
| Scale item | Scale | IDR | Day 7 | at Day 7 | improvement |
| Reduced Sleep | MADRS | 25 | 9 | 16 | 64.00% |
| Concentration Difficulties | MADRS | 30 | 9 | 21 | 70.00% |
| Lassitude | MADRS | 27 | 3 | 24 | 88.89% |
| Inability to Feel | MADRS | 36 | 6 | 30 | 83.33% |
| Inner Tension | MADRS | 26 | 12 | 14 | 53.85% |
| Pessimistic Thoughts | MADRS | 28 | 3 | 25 | 89.29% |
| Suicidal Thoughts | MADRS | 11 | 3 | 8 | 72.73% |
| Guilt feelings | BPRS | 34 | 10 | 24 | 70.59% |
| Tension | BPRS | 16 | 10 | 6 | 37.50% |
| Anxiety | BPRS | 37 | 17 | 20 | 54.05% |
| Emotional withdrawal | BPRS | 13 | 8 | 5 | 38.46% |
| Blunted affect | BPRS | 15 | 8 | 7 | 46.67% |

TABLE 3-A
Scores recorded against relevant MADRS and BPRS items for patients assigned
to 12 mg dosing regimen. The item scores represent the sum of the individual
patient scores for all patients (n = 4) in the 12 mg group. Assessment
2 hours (MADRS) or 3 hours (BPRS) after administration of the dose.

| | | | | Improvement | |
|---|---|---|---|---|---|
| | | Baseline | 2/3 | after 2/3 | Percentage |
| Scale item | Scale | 12 mg | hours | hours | improvement |
| Concentration Difficulties | MADRS | 16 | 7 | 9 | 56.25% |
| Lassitude | MADRS | 16 | 10 | 6 | 37.50% |
| Inability to Feel | MADRS | 16 | 9 | 7 | 43.75% |
| Inner Tension | MADRS | 13 | 2 | 11 | 84.62% |
| Pessimistic Thoughts | MADRS | 16 | 8 | 8 | 50.00% |
| Suicidal Thoughts | MADRS | 8 | 3 | 5 | 62.50% |
| Guilt Feelings | BPRS | 18 | 9 | 9 | 50.00% |
| Tension | BPRS | 14 | 9 | 5 | 35.71% |
| Anxiety | BPRS | 25 | 11 | 14 | 56.00% |
| Emotional Withdrawal | BPRS | 13 | 11 | 2 | 15.38% |
| Blunted Affect | BPRS | 11 | 8 | 3 | 27.27% |

TABLE 3-B
Scores recorded against relevant MADRS and BPRS items for patients assigned to
12 mg dosing regimen. The item scores represent the sum of the individual patient
scores for all patients (n = 4) in the 12 mg group. Assessment on day 1.

| | | Baseline | | Improvement | Percentage |
|---|---|---|---|---|---|
| Scale item | Scale | 12 mg | Day 1 | at Day 1 | improvement |
| Reduced Sleep | MADRS | 12 | 10 | 2 | 16.67% |
| Concentration Difficulties | MADRS | 16 | 2 | 14 | 87.50% |
| Lassitude | MADRS | 16 | 0 | 16 | 100.00% |
| Inability to Feel | MADRS | 16 | 1 | 15 | 93.75% |
| Inner Tension | MADRS | 13 | 3 | 10 | 76.92% |
| Pessimistic Thoughts | MADRS | 16 | 7 | 9 | 56.25% |
| Suicidal Thoughts | MADRS | 8 | 5 | 3 | 37.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 8 | 5 | 38.46% |
| Blunted Affect | BPRS | 11 | 6 | 5 | 45.45% |

TABLE 3-C
Scores recorded against relevant MADRS and BPRS items for patients assigned to
12 mg dosing regimen. The item scores represent the sum of the individual patient
scores for all patients (n = 4) in the 12 mg group. Assessment on day 7.

| | | Baseline | | Improvement | Percentage |
|---|---|---|---|---|---|
| Scale item | Scale | 12 mg | Day 7 | at Day 7 | improvement |
| Reduced Sleep | MADRS | 12 | 6 | 6 | 50.00% |
| Concentration Difficulties | MADRS | 16 | 3 | 13 | 81.25% |
| Lassitude | MADRS | 16 | 3 | 13 | 81.25% |
| Inability to Feel | MADRS | 16 | 1 | 15 | 93.75% |
| Inner Tension | MADRS | 13 | 5 | 8 | 61.54% |
| Pessimistic Thoughts | MADRS | 16 | 8 | 8 | 50.00% |
| Suicidal Thoughts | MADRS | 8 | 7 | 1 | 12.50% |
| Guilt feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional withdrawal | BPRS | 13 | 6 | 7 | 53.85% |
| Blunted affect | BPRS | 11 | 5 | 6 | 54.55% |

### Example 6—Assessment of the Pharmacokinetics of 5-MeO-DMT and Bufotenine

In order to investigate the pharmacokinetic properties of 5-MeO-DMT, three groups of 8 subjects each were formed. Subjects were administered a single dose of 6 mg; 12 mg or 18 mg 5-MeO-DMT via inhalation. Blood samples were obtained at 1; 2; 4; 7; 10; 15; 20; 30; 45 min and 1; 1.5; 2; 3; 4 hours after administration.

5-MeO-DMT concentrations were determined using LC-MS/MS. PK parameters were generated by algebraic analysis of the concentration versus time plots for each individual. The analysis was carried out using the software Phoenix WinNonlin 6.3.

Median Cmax values obtained for the three groups were 11.85 ng/ml (6 mg group), 22.90 ng/ml (12 mg group) and 38.45 ng/ml (18 mg group).

Table 4 below shows median percentage plasma concentrations relative to Cmax as determined for the time points indicated.

| | | Median percentage plasma concentration relative to Cmax | | | | | | | | | | | | | |
|---|---|---|---|---|---|---|---|---|---|---|---|---|---|---|---|
| | | time (min) | | | | | | | | | | | | | |
| | | 1 | 2 | 4 | 7 | 10 | 15 | 20 | 30 | 45 | 60 | 90 | 120 | 180 | 240 |
| 6 | mg | 96 | 98 | 83 | 56 | 40 | 22 | 16 | 10 | 8 | 4 | 2 | 1 | 0 | N.A. |
| 12 | mg | 100 | 79 | 59 | 30 | 22 | 12 | 8 | 5 | 3 | 2 | 0 | 0 | 0 | 0 |
| 18 | mg | 100 | 79 | 59 | 30 | 22 | 12 | 8 | 5 | 3 | 2 | 0 | 0 | 0 | 0 |

Pharmacokinetic measurements were also carried out for dosing schemes relying on uptitration. Substantially similar results were obtained.

Blood concentrations were also determined for the 5-MeO-DMT metabolite bufotenine. Only in few samples, concentrations were above the lower level of quantification (LLOQ) (25 pg/ml). From 15 min onwards, the bufotenine concentration was always below the LLOQ.

Substantially similar observations were made when subjects receiving an uptitration scheme were included.

### Example 7—Toxicological Testing of 5-MeO-DMT

5-MeO-DMT did not induce mutation in four histidine-requiring bacterial strains (TA98, TA100, TA1535 and TA1537) of Salmonella typhimurium, and one tryptophan-requiring strain (WP2 uvrA pKM101) of Escherichia coli. These conditions included treatments at concentrations up to 5000 μg/plate (the maximum recommended concentration according to current regulatory guidelines), in the absence and in the presence of a rat liver metabolic activation system (S-9).

### Example 8-5-MeO-DMT Binding to Human Plasma Proteins

The in vitro binding of 5-MeO-DMT to plasma proteins was determined using high throughput dialysis. Equilibration time and non-specific binding were determined at a nominal 5-MeO-DMT concentration of 1 μM using human plasma. Following evaluation of the equilibration data, plasma protein binding was investigated at nominal concentrations of 0.1, 1 and 10 μM using a 4-hour dialysis time. The concentration of 5-MeO-DMT in the samples from the plasma and buffer compartments was determined by LC-MS/MS. The protein binding results are presented below:

TABLE 5
Percentage of free fractions of 5-MeO-DMT in human plasma

| 5-MeO-DMT (μM) | Free Fraction, % |
|---|---|
| 0.1 | 78.7 |
| 1 | 77.3 |
| 10 | 87.0 |

### Example 9—Human Metabolism of 5-MeO-DMT

5-MeO-DMT was incubated at a nominal concentration of 1 μM and 10 μM with human hepatocytes in suspension in Leibovitz L-15 medium (1×106 cells/mL).

A standard stock solution of 5-MeO-DMT was prepared in ethanol at 20 mM and was further diluted with Leibovitz L-15 medium to a concentration of 2 mM. For incubations with cryopreserved hepatocytes, the 2 mM stock solution was diluted with Leibovitz L-15 medium to a concentration of 20 μM or 2 μM. An aliquot (250 μL) of the 20 μM and 2 μM test substance formulations was added to each hepatocyte incubation sample (250 μL), as appropriate, so that the final test substance concentration in the incubations was 10 μM or 1 μM, respectively, and incubations contained less than 1% (v/v) solvent.

Incubations were performed at ca. 37° C. in a shaking water bath (total incubation volume 0.5 mL). For 1 μM, incubations were terminated after 0, 5, 10, 20, 30, 60 and 120 minutes, by the addition of ice-cold acetonitrile (0.5 mL). For 10 μM, incubations were terminated after 0, 10, 30, 60 and 120 minutes, by the addition of ice-cold acetonitrile containing internal standard (1 μg/mL Psilocin-d10).

The samples were vortex mixed and centrifuged at ca 13,000 rpm for 10 minutes at room temperature. Following centrifugation, the protein-free supernatants were removed for analysis.

Blank control incubations were carried out with Leibovitz L-15 medium in place of the test substance. No cells control samples were performed with Leibovitz L-15 medium in place of hepatocytes. Aliquots of the blank control samples were taken at 120 minutes, while no cells control samples were taken at 0, 30 and 120 for 1 μM incubations and at 0 and 120 minutes for 10 μM incubations.

All 1 μM incubations were performed in duplicate, while all 10 μM incubations were performed in singlet. All samples were stored at −80° C. (nominal) prior to analysis.

Suitable chromatographic conditions were developed to retain the parent compound and give a suitable chromatographic response. The 0, 30 and 120-minute incubation samples generated following incubation of 5-MeO-DMT at 10 μM were analysed using reverse phase LC-MS analysis to generate high and low energy mass spectra (MSE). Prior to sample analysis a 100 μL aliquot of each sample was evaporated to near dryness under a steady stream of nitrogen at room temperature, and subsequently reconstituted in 50 μL of mobile phase A (0.1% formic acid in water). Each sample (0, 30 and 120-minute, 10 μM) was analysed using accurate mass LC-MS to determine relative levels of parent compound at each time-point, and determine the profile of metabolites formed. Appropriate blank and control samples were also analysed. The 10 and 60-minute, 10 μM incubation samples were not analysed and were stored at −80° C. (nominal).

Data were interrogated for the presence of metabolites by comparison of retention times with the test substance reference standard and based on the accurate masses of potential metabolites using screening software (UNIFI version 1.9.4), and user defined search parameters. To confirm a suspected metabolite, the measured accurate mass of the peak detected in the sample used for structural elucidation had to be within 5 ppm of the theoretical mass in order to confirm the molecular formula.

Results obtained are summarized in the above table 1.

### Example 10—Metabolic Stability for 5-Methoxy Indole-3-Acetic Acid (5-MIAA) and 5-Methoxytryptophol in a Human Hepatocyte Co-Culture Model

The metabolic stability of 5-MIAA and 5-methoxytryptophol was investigated in a Hμrel co-culture assay with human hepatocytes (Hμrel HumanPool™, primary hepatic co-culture model from Visikol Inc.).

The incubations were performed using 1 and 10 μM initial concentrations and sampling at 0, 1, 2, 4, 8, 24, 48, and 72 hours (h) time points. The samples were analysed using UPLC/QE-orbitrap-MS.

The remaining LC/MS peak areas detected for test compounds after each incubation time point with Hμrel co-culture assay, relative to corresponding 0 min incubation samples, are shown in the tables below. Results (disappearance half-lives) for the assay control diazepam indicated that enzyme activities were within the normal level.

TABLE 6
The relative LC/MS peak areas for 5-MIAA after 0-72 h incubations. The initial
substrate concentrations were 1 and 10 μM (n = 2 for 1 μM and n = 1 for 10 μM).

| | Incubation | | | | | | | |
|---|---|---|---|---|---|---|---|---|
| | 0 h, % | 1 h, % | 2 h, % | 4 h, % | 8 h, % | 24 h, % | 48 h, % | 72 h, % |
| 1 μM Hepatocytes | 100 | 98 | 100 | 99 | 95 | 99 | 88 | 82 |
| 1 μM Stromal cells | 100 | — | — | 103 | 107 | 108 | 111 | 135 |
| 10 μM Hepatocytes | 100 | 97 | 100 | 103 | 96 | 96 | 86 | 75 |
| 10 μM Stromal cells | 100 | — | — | 101 | 106 | 97 | 103 | 115 |

TABLE 7
The relative LC/MS peak areas for 5-methoxytryptophol after 0-72 h incubations. The initial
substrate concentrations were 1 and 10 μM (n = 2 for 1 μM and n = 1 for 10 μM).

| | Incubation | | | | | | | |
|---|---|---|---|---|---|---|---|---|
| | 0 h, % | 1 h, % | 2 h, % | 4 h, % | 8 h, % | 24 h, % | 48 h, % | 72 h, % |
| 1 μM Hepatocytes | 100 | 79 | 57 | 33 | 10 | 0 | 0 | 0 |
| 1 μM Stromal cells | 100 | — | — | 97 | 107 | 94 | 102 | 104 |
| 10 μM Hepatocytes | 100 | 87 | 81 | 68 | 42 | 1 | 0 | 0 |
| 10 μM Stromal cells | 100 | — | — | 99 | 105 | 86 | 98 | 104 |

A low metabolic turnover was observed for 5-MIAA, the remaining abundances after 72 h period being 75-82% in the presence of hepatocytes, while no disappearance was observed with stromal cell controls.

For 5-methoxytryptophol, a high metabolic turnover was observed, with complete disappearance in 24 h in the presence of hepatocytes, and no disappearance with stromal cell controls.

With human hepatocytes and 1 μM test concentration, in vitro intrinsic clearance of 0.15 μl/min/million cells (half-life 15 400 min) was obtained for 5-MIAA, while the corresponding value for 5-methoxytryptophol was 16.2 μl/min/million cells (half-life 142 min).

The predicted hepatic extraction ratios were 2% for 5-MIAA and 67% 5-methoxytryptophol.

### Example 11—Plasma Binding of 5-MIAA

Binding to human plasma protein was determined. Reported are the unbound fraction (fu) for three replicates as well as the mean unbound fraction, the standard deviation and the mean % recovery (Table 8).

| | Repli- | Repli- | Repli- | Mean | | Mean % |
|---|---|---|---|---|---|---|
| Compound | cate 1 | cate 2 | cate 3 | fu | SD | Recovery |
| 5-MIAA | 0.499 | 0.462 | 0.544 | 0.502 | 0.0411 | 81.4 |
| Warfarin | 0.0418 | 0.0356 | 0.0358 | 0.0377 | 0.00352 | 78.1 |
| (control) | | | | | | |

### Example 12—Clinical Trial of 5-MeO-DMT Administered Via Inhalation to Patients with Postpartum Depression

The single-arm, open-label clinical trial will involve 15 adult female patients with clinically diagnosed postpartum depression (PPD).

The patients will receive a single-day individualized 5-MeO-DMT dosing regimen via inhalation after vaporization.

More in particular, the patients will receive up to three doses of 5-MeO-DMT on Day 0: 6 mg, 12 mg, and 18 mg.
1. All patients will receive an initial dose of 6 mg 5-MeO-DMT.
2. The second dose (12 mg) will only be administered if:
   a. A peak experience (total score of •75) has not been achieved following the 6 mg dose, and
   b. The 6 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (18 mg) will only be administered if:
   a. A peak experience (total score of •75) has not been achieved following the 12 mg dose, and
   b. The 12 mg dose was safe and well-tolerated according to the investigator, and
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing. Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate<1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, •uncontrolled•hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's-judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 6 mg, 12 mg and 18 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 6 mg, 12 mg and 18 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day DR of 6 mg, 12 mg and 18 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS• 10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (•50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score •75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing.

One patient with postpartum depression diagnosed by a psychiatrist has, so far, been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient was diagnosed with postpartum depression after giving birth to her third child. The patient completed all planned visit days. The inhalation procedure was adequately performed by the patient and was well tolerated with no inhalation-related adverse events.

### Results

Except for a temporary, clinically non-relevant increase in heart rate and blood pressure shortly after administration of 5-MeO-DMT, no other noteworthy changes in vital parameters occurred. Assessments of ECG (at 3 hours after administration) and safety laboratory analyses (at 7 days), CADSS (at 3 hours, 1 day and 7 days) were unremarkable. The few reported adverse events (cramping left abdominal pain and headache, both on Day 0) were mild, short-lasting and resolved spontaneously by the end of the study.

With regard to the intensity of the psychedelic experience, the recorded PES score achieved upon exposure to a nominal dose of 6 mg was 17.3. This score indicated the need to proceed to the administration of a subsequent, higher dose of 12 mg, per the design of the individualised dosing regimen. The PES score achieved for this dose was 85.7 and, being •75, indicated the occurrence of a peak psychedelic experience and the completion of the IDR for this patient.

Significantly, the patient reported a major improvement in her depressive symptoms as assessed by MADRS at the earliest assessment timepoint of 2 hours after drug administration, with the effect being maintained over time (Table 9). The patient also fulfilled standard criteria for MADRS response (at least 50% improvement from baseline) and MADRS remission (MADRS total score equal or less than 10).

TABLE 9
MADRS/BPRS scores table

| | | 2 hr | | | Improvement | | |
|---|---|---|---|---|---|---|---|
| | | (MADRS); | | | 2 hr; | | |
| | Base- | Discharge | | | Dis- | | |
| Scale item | line | (BPRS) | Day 1 | Day 7 | charge | Day 1 | Day 7 |
| MADRS | | | | | | | |
| Reported sadness | 4 | 0 | 0 | 0 | 4 | 4 | 4 |
| Apparent sadness | 5 | 0 | 0 | 0 | 5 | 5 | 5 |
| Inner Tension | 5 | 0 | 0 | 1 | 5 | 5 | 4 |
| Reduced Sleep | 5 | 5 | 0 | 0 | 0 | 5 | 5 |
| Reduced appetite | 0 | 0 | 0 | 0 | 0 | 0 | 0 |
| Concentration Difficulties | 0 | 0 | 0 | 0 | 0 | 0 | 0 |
| Lassitude | 3 | 0 | 0 | 0 | 3 | 3 | 3 |
| Inability to Feel | 3 | 0 | 0 | 0 | 3 | 3 | 3 |
| Pessimistic Thoughts | 4 | 0 | 0 | 0 | 4 | 4 | 4 |
| Suicidal Thoughts | 0 | 0 | 0 | 0 | 0 | 0 | 0 |
| Total MADRS | 29 | 5 | 0 | 1 | 24 | 29 | 28 |
| BPRS | | | | | | | |
| Somatic Concern | 6 | 1 | 1 | 1 | 5 | 5 | 5 |
| Anxiety | 7 | 1 | 1 | 1 | 6 | 6 | 6 |
| Emotional withdrawal | 5 | 1 | 1 | 1 | 4 | 4 | 4 |
| Conceptual disorganization | 1 | 1 | 1 | 1 | 0 | 0 | 0 |
| Guilt feelings | 6 | 1 | 1 | 1 | 5 | 5 | 5 |
| Tension | 6 | 1 | 1 | 1 | 5 | 5 | 5 |
| Mannerisms and Posturing | 1 | 1 | 1 | 1 | 0 | 0 | 0 |
| Grandiosity | 1 | 1 | 1 | 1 | 0 | 0 | 0 |
| Depressive Mood | 6 | 1 | 1 | 1 | 5 | 5 | 5 |
| Hostility | 1 | 1 | 1 | 1 | 0 | 0 | 0 |
| Suspiciousness | 1 | 1 | 1 | 1 | 0 | 0 | 0 |
| Hallucinatory behaviour | 1 | 1 | 1 | 1 | 0 | 0 | 0 |
| Motor retardation | 1 | 1 | 1 | 1 | 0 | 0 | 0 |
| Uncooperativeness | 1 | 1 | 1 | 1 | 0 | 0 | 0 |
| Unusual Thought Content | 1 | 1 | 1 | 1 | 0 | 0 | 0 |
| Blunted affect | 1 | 1 | 1 | 1 | 0 | 0 | 0 |
| Excitement | 2 | 1 | 1 | 1 | 1 | 1 | 1 |
| Disorientation | 1 | 1 | 1 | 1 | 0 | 0 | 0 |

Significant improvements were noted for several MADRS items in particular. The items are outlined in Table 9. While the patient's baseline scores for some items reflected absence of the symptom (reduced appetite, concentration difficulties, suicidal thoughts), items with scores reflecting severe symptoms (e.g., reduced sleep, inner tension) saw remarkable improvement.

Similarly, improvements were seen in several BPRS items, including Somatic Concerns, Anxiety, Emotional withdrawal, Guilt feelings and Tension.

Additionally, improvements in maternal functioning were evidenced by improvements in the BIMF score recorded at Day 7, as outlined in Table 10, with the total score improving by 14% from 92 to 105 (out of a possible total of 120).

Several functional domains of maternal function were also assessed, as defined by Barkin et al. The improvements in each functional domain are outlined in more detail in Table 11.

Here, noteworthy improvements in self-care, psychological well-being and management were achieved, with percentage improvements ranging from 18% (management) to (44%),% (self-care). These improvements reinforce the relationship between improvement in depressive items, as assessed by the MADRS, and improvements in maternal functioning.

It is noted that the patient scored comparatively high already before treatment. In some functional domains, the score was at the maximum value, or close to it (see Table 11), so that the scope for improvement by therapy was limited.

TABLE 10
BIMF scores table

| No. | BIM F item | Day 0 | Day 7 | Δ (Day 7) |
|---|---|---|---|---|
| 1 | I am a good mother | 6 | 6 | 0 |
| 2 | I feel rested | 0 | 2 | 2 |
| 3 | I am comfortable with the way I've chosen to feed my | 6 | 6 | 0 |
| | baby (either bottle or breast, or both) | | | |
| 4 | My baby and I understand each other | 5 | 5 | 0 |
| 5 | I am able to relax and enjoy time with my baby | 5 | 6 | 1 |
| 6 | There are people in my life that I can trust to care for | 6 | 6 | 0 |
| | my baby when I need a break | | | |
| 7 | I am comfortable allowing a trusted friend or relative | 6 | 6 | 0 |
| | to care for my baby (can include baby's father or partner) | | | |
| 8 | I am getting enough adult interaction | 6 | 6 | 0 |
| 9 | I am getting enough encouragement from other people | 5 | 6 | 1 |
| 10 | I trust my own feelings (instincts) when it comes to | 5 | 6 | 1 |
| | taking care of my baby | | | |
| 11 | I take a little time each week to do something for myself | 5 | 6 | 1 |
| 12 | I am taking good care of my baby's physical needs | 6 | 6 | 0 |
| | (feedings, changing diapers, doctor's appointments) | | | |
| 13 | I am taking good care of my physical needs (eating, | 4 | 5 | 1 |
| | showering, etc) | | | |
| 14 | I make good decisions about my baby's health and | 6 | 6 | 0 |
| | well being | | | |
| 15 | My baby and I are getting into a routine | 5 | 6 | 1 |
| 16 | I worry about how other people judge me (as a | 3 | 5 | 2 |
| | mother) | | | |
| 17 | I am able to take care of my baby and my other | 5 | 5 | 0 |
| | responsibilities | | | |
| 18 | Anxiety or worry often interferes with my mothering | 2 | 5 | 3 |
| | ability | | | |
| 19 | As time goes on, I am getting better at taking care of | 0 | 1 | 1 |
| | my baby | | | |
| 20 | I am satisfied with the job I am doing as a new mother | 6 | 5 | −1 |
| | TOTAL | 92 | 105 | 13 |

TABLE 11
BIMF functional domain scores table

| | | Score | | | | |
|---|---|---|---|---|---|---|
| | Related | Maxi- | | | Δ | % |
| Functional | BIMF | mum | Day | Day | (Day | Improve- |
| Domain | Items | Possible | 0 | 7 | 7) | ment |
| Self-Care | 2, 11, 13 | 18 | 9 | 13 | 4 | 44.44% |
| Infant Care | 12, 14 | 12 | 12 | 12 | 0 | 0.00% |
| Mother-Child | 4, 5, 15 | 18 | 15 | 17 | 2 | 13.33% |
| Interaction | | | | | | |
| Psychological | 1, 2, 3, 5, | 60 | 44 | 53 | 9 | 20.45% |
| Well-being | 7, 10, 11, | | | | | |
| | 16, 18, 20 | | | | | |
| Social Support | 6, 8, 9 | 18 | 17 | 18 | 1 | 5.88% |
| Management | 7, 11, 13, | 36 | 28 | 33 | 5 | 17.86% |
| | 14, 17, 18 | | | | | |
| Adjustment | 17, 19 | 12 | 5 | 6 | 1 | 20.00% |

### SUMMARY AND CONCLUSIONS

A. An individualised dosing regimen of 6 mg 5-MeO-DMT, followed by 12 mg 5-MeO-DMT administered via inhalation was well tolerated and induced an astonishing and very significant clinical response in a patient formally diagnosed with postpartum depression.
B. The clinical response occurs rapidly within 2 hours after 5-MeO-DMT administration. Such rapid onset is unusual and has not been seen with conventional classes of antidepressants, including tricyclic antidepressants, monoamine oxidase inhibitors, selective serotonin-reuptake inhibitors (SSRIs), serotonin-norepinephrine reuptake inhibitors (SNRIs), and others, which generally take 4 to 6 weeks to show their effect.
C. The patient experienced a clinical remission within 2 hours after 5-MeO-DMT administration according to the IDR. This is highly superior to any approved therapy for postpartum depression, and also to all previously tested psychedelic agents.
D. A significant clinical response was sustained over the 7-day follow-up period, although 5-MeO-DMT was only given once and is no longer efficaciously present in the body during this time frame (see pharmacokinetic data in Example 6 above). This observation supports the superior clinical profile of 5-MeO-DMT and allows for convenient administration intervals.
E. In addition to anti-depressive effects, endpoints assessing other symptoms (such as somatic concerns, emotional withdrawal, anxiety, guilt and tension) were positively impacted, supporting the use of 5-MeO-DMT in patients with other mental diseases.
F. In addition to anti-depressive effects, endpoints assessing maternal functioning, as assessed using the BIMF, such as self-care, psychological well-being and management, were positively impacted. This supports additional benefits of 5-MeO-DMT to patients suffering from PPD beyond improvement in their core depressive symptoms.

The highlighted aspects show that 5-MeO-DMT has a significantly improved efficacy profile compared to approved pharmacological therapies for postpartum depression and to all previously tested psychedelic agents, when used according to the present invention.

Together with the short duration of the acute psychedelic effects and the favourable safety profile, these data show that the technical problem to provide an improved psychoactive therapy in a patient with a postpartum depression is solved by the present invention.

1. 5-Methoxy-N,N-dimethyltryptamine (5-MeO-DMT) or a pharmaceutically acceptable salt thereof for use in treating a patient suffering from postpartum depression (PPD).

2. 5-MeO-DMT or a pharmaceutically acceptable salt thereof for use as in claim 1, wherein the patient has a Montgomery-Åsberg Depression Rating Scale (MADRS) score of 20 or more or a 17-item Hamilton Depression Rating Scale (HAM-D) score of 16 or more.

3. 5-MeO-DMT or a pharmaceutically acceptable salt thereof for use as in claim 2, wherein the patient has a MADRS score of 28 or more or a HAM-D score of 22 or more.

4. 5-MeO-DMT or a pharmaceutically acceptable salt thereof for use as in claim 2, wherein the patient has a MADRS score of 35 or more or by a HAM-D score of 27 or more.

5. 5-MeO-DMT or a pharmaceutically acceptable salt thereof for use as in claims 1 to 4, wherein the patient is diagnosed with a treatment-resistant form of postpartum depression.

6. 5-MeO-DMT or a pharmaceutically acceptable salt thereof for use as in claims 1 to 5, wherein the patient suffers in addition from suicidal ideation.

7. 5-MeO-DMT or a pharmaceutically acceptable salt thereof for use as in claims 1 to 6, wherein the patient suffers in addition from slightly compromised, compromised or severely compromised maternal functioning.

8. 5-MeO-DMT or a pharmaceutically acceptable salt thereof for use as in claim 7, wherein the patient has a Barkin Index of Maternal Functioning (BIMF) score of 95 or below such as 80 or below, in particular 65 or below.

9. 5-MeO-DMT or a pharmaceutically acceptable salt thereof for use as in claims 1 to 8, wherein the 5-MeO-DMT or salt thereof is administered at a dose or in a dosage regimen that causes the patient to experience a peak psychedelic experience.

10. 5-MeO-DMT or a pharmaceutically acceptable salt thereof for use as in claims 1 to 9, wherein a dosage of about 4 mg to about 20 mg 5-MeO-DMT is administered, or wherein equimolar amounts of the pharmaceutically acceptable salt are administered instead of 5-MeO-DMT.

11. 5-MeO-DMT or a pharmaceutically acceptable salt thereof for use as in claims 1 to 9, wherein a dosage of about 6 mg; or of about 12 mg; or of about 18 mg is administered, or wherein equimolar amounts of the pharmaceutically acceptable salt are administered instead of 5-MeO-DMT.

12. 5-MeO-DMT or a pharmaceutically acceptable salt thereof for use as in claims 1 to 10, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

13. 5-MeO-DMT or a pharmaceutically acceptable salt thereof for use as in claims 1 to 12, wherein the 5-MeO-DMT is administered in a dosage from about 2 mg to about 8 mg for a first administration, and then increased, unless the patient has already experienced a peak psychedelic experience, to a dosage from about 8 mg to about 14 mg for a second administration, and then increased, unless the patient has already experienced a peak psychedelic experience, to a dosage from about 14 mg to about 20 mg for a third administration, or wherein equimolar amounts of the pharmaceutically acceptable salt are administered instead of 5-MeO-DMT.

14. 5-MeO-DMT or a pharmaceutically acceptable salt thereof for use as in claim 13, wherein the first dosage of 5-MeO-DMT is about 6 mg, the second dosage of 5-MeO-DMT is about 12 mg, and the third dosage of 5-MeO-DMT is about 18 mg; or wherein equimolar amounts of the pharmaceutically acceptable salt are administered instead of 5-MeO-DMT.

15. 5-MeO-DMT or a pharmaceutically acceptable salt thereof for use as in claims 12 to 14, wherein the interval between two administrations is not less than 1 hour and not more than 24 hours, such as about 1 to 4 hours, preferably about 1 to 2 hours.

16. 5-MeO-DMT or a pharmaceutically acceptable salt thereof for use as in claims 9 to 15, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

17. 5-MeO-DMT or a pharmaceutically acceptable salt thereof for use as in claim 16, wherein the occurrence of a peak psychedelic experience is identified through achievement of a Peak Experience Scale (PES) Total Score of at least 75.

18. 5-MeO-DMT or a pharmaceutically acceptable salt thereof for use as in claims 1 to 17, wherein the 5-MeO-DMT or a pharmaceutically acceptable salt thereof is administered via inhalation or by nasal, buccal or sublingual administration.

19. 5-MeO-DMT or a pharmaceutically acceptable salt thereof for use as in claim 18, wherein 5-MeO-DMT or a pharmaceutically acceptable salt thereof is administered in the form of an aerosol comprising (a) a pharmaceutically acceptable gas; (b) aerosol particles of 5-methoxy-N,N-dimethyltryptamine (5-MeO-DMT) or a pharmaceutically acceptable salt thereof, wherein the aerosol has an aerosol particle mass density of about 0.5 mg/l to about 18 mg/l, such as to about 12.5 mg/l.

20. 5-MeO-DMT or a pharmaceutically acceptable salt thereof for use as in claim 19, wherein the aerosol is generated by a) exposing a thin layer of 5-MeO-DMT or a pharmaceutically acceptable salt thereof, configured on a solid support, to thermal energy, and b) passing air over the thin layer to produce aerosol particles.

21. 5-MeO-DMT or a pharmaceutically acceptable salt thereof for use as in claims 18 to 20, wherein the dosage amount of 5-MeO-DMT or a pharmaceutically acceptable salt to be administered to the patient is inhaled with a single breath.

22. 5-MeO-DMT for use as in claims 18 to 21, wherein the 5-MeO-DMT is used in the form of the free base.

23. 5-MeO-DMT or a pharmaceutically acceptable salt thereof for use as in claims 1 to 22, wherein a clinical response, as reflected by a reduction in the Clinical Global Impression-Severity (CGI-S) score, occurs not later than about 2 hours after the last administration of 5-MeO-DMT or a pharmaceutically acceptable salt thereof.

24. 5-MeO-DMT or a pharmaceutically acceptable salt thereof for use as in claims 1 to 23, wherein a clinical response, as reflected by a reduction in the Clinical Global Impression-Severity (CGI-S) score, occurs on day 1, for instance, about 24 hours after the last administration of 5-MeO-DMT or a pharmaceutically acceptable salt thereof.

25. 5-MeO-DMT or a pharmaceutically acceptable salt thereof for use as in claims 1 to 24, wherein a clinical response, as reflected by a reduction in the Clinical Global Impression-Severity (CGI-S) score, persists until at least 6 days after the last administration of 5-MeO-DMT or a pharmaceutically acceptable salt thereof.

26. 5-MeO-DMT or a pharmaceutically acceptable salt thereof for use as in claims 1 to 25, wherein a clinical response, as reflected by a reduction in the Clinical Global Impression-Severity (CGI-S) score, persists until at least 14 days after the last administration of 5-MeO-DMT or a pharmaceutically acceptable salt thereof.

27. 5-MeO-DMT or a pharmaceutically acceptable salt thereof for use as in claims 1 to 26, wherein a clinical response, as reflected by a reduction in the Clinical Global Impression-Severity (CGI-S) score, persists until at least 28 days after the last administration of 5-MeO-DMT or a pharmaceutically acceptable salt thereof.

28. 5-MeO-DMT or a pharmaceutically acceptable salt thereof for use as in claims 1 to 27, wherein a clinical response, as assessed by at least 50% improvement of the MADRS or HAM-D score, compared to the respective score prior to treatment, occurs not later than about 2 hours after the last administration of 5-MeO-DMT or a pharmaceutically acceptable salt thereof.

29. 5-MeO-DMT or a pharmaceutically acceptable salt thereof for use as in claims 1 to 28, wherein a clinical response, as assessed by at least 75% improvement of the MADRS or HAM-D score, compared to the respective score prior to treatment, occurs not later than about 2 hours after the last administration of 5-MeO-DMT or a pharmaceutically acceptable salt thereof.

30. 5-MeO-DMT or a pharmaceutically acceptable salt thereof for use as in claims 1 to 29, wherein a remission of depressive symptoms, as assessed by a MADRS score equal to or less than 10, or a HAM-D score equal to or less than 7, occurs not later than about 2 hours after the last administration of 5-MeO-DMT or a pharmaceutically acceptable salt thereof.

31. 5-MeO-DMT or a pharmaceutically acceptable salt thereof for use as in claims 1 to 30, wherein a remission of depressive symptoms, as assessed by a MADRS score equal to or less than 10, or a HAM-D score equal to or less than 7, occurs on day 1, for instance, about 24 hours after the last administration of 5-MeO-DMT or a pharmaceutically acceptable salt thereof.

32. 5-MeO-DMT or a pharmaceutically acceptable salt thereof for use as in claims 1 to 31, wherein the clinical response, as assessed by at least 50% improvement of the MADRS or HAM-D score, compared to the respective score prior to treatment, persists until at least 6 days after the last administration of 5-MeO-DMT or a pharmaceutically acceptable salt thereof.

33. 5-MeO-DMT or a pharmaceutically acceptable salt thereof for use as in claims 1 to 32, wherein there is a clinical response, as assessed by at least 75% improvement of the MADRS or HAM-D score, compared to the respective score prior to treatment, on day 7 after the last administration of 5-MeO-DMT or a pharmaceutically acceptable salt thereof.

34. 5-MeO-DMT or a pharmaceutically acceptable salt thereof for use as in claims 1 to 33, wherein the patient is in remission of depressive symptoms, as assessed by a MADRS score equal to or less than 10, or a HAM-D score equal to or less than 7, on day 7 after the last administration of 5-MeO-DMT or a pharmaceutically acceptable salt thereof.

35. 5-MeO-DMT or a pharmaceutically acceptable salt thereof for use as in claims 1 to 34, wherein the clinical response, as assessed by at least 50% improvement of the MADRS or HAM-D score, compared to the respective score prior to treatment, persists until at least 14 days after the last administration of 5-MeO-DMT or a pharmaceutically acceptable salt thereof.

36. 5-MeO-DMT or a pharmaceutically acceptable salt thereof for use as in claims 1 to 35, wherein there is a clinical response, as assessed by at least 75% improvement of the MADRS or HAM-D score, compared to the respective score prior to treatment, on day 14 after the last administration of 5-MeO-DMT or a pharmaceutically acceptable salt thereof.

37. 5-MeO-DMT or a pharmaceutically acceptable salt thereof for use as in claims 1 to 36, wherein the patient is in remission of depressive symptoms, as assessed by a MADRS score equal to or less than 10, or a HAM-D score equal to or less than 7, on day 14 after the last administration of 5-MeO-DMT or a pharmaceutically acceptable salt thereof.

38. 5-MeO-DMT or a pharmaceutically acceptable salt thereof for use as in claims 1 to 37, wherein the clinical response, as assessed by at least 50% improvement of the MADRS or HAM-D score, compared to the respective score prior to treatment, persists until at least 28 days after the last administration of 5-MeO-DMT or a pharmaceutically acceptable salt thereof.

39. 5-MeO-DMT or a pharmaceutically acceptable salt thereof for use as in claims 1 to 38, wherein there is a clinical response, as assessed by at least 75% improvement of the MADRS or HAM-D score, compared to the respective score prior to treatment, on day 28 after the last administration of 5-MeO-DMT or a pharmaceutically acceptable salt thereof.

40. 5-MeO-DMT or a pharmaceutically acceptable salt thereof for use as in claims 1 to 39, wherein the patient is in remission of depressive symptoms, as assessed by a MADRS score equal to or less than 10, or a HAM-D score equal to or less than 7, on day 28 after the last administration of 5-MeO-DMT or a pharmaceutically acceptable salt thereof.

41. 5-Methoxy-N,N-dimethyltryptamine (5-MeO-DMT) or a pharmaceutically acceptable salt thereof for use in any of claims 1 to 40, wherein the patient is a breastfeeding mother who is advised to discontinue breastfeeding until 48 hours after the last administration of 5-MeO-DMT or a pharmaceutically acceptable salt thereof.

42. 5-Methoxy-N,N-dimethyltryptamine (5-MeO-DMT) or a pharmaceutically acceptable salt thereof for use in any of claims 1 to 40, wherein the patient is a breastfeeding mother who is advised to discontinue breastfeeding until 24 hours after the last administration of 5-MeO-DMT or a pharmaceutically acceptable salt thereof.

43. 5-Methoxy-N,N-dimethyltryptamine (5-MeO-DMT) or a pharmaceutically acceptable salt thereof for use in any of claims 1 to 40, wherein the patient is a breastfeeding mother who is advised to discontinue breastfeeding until 6 hours after the last administration of 5-MeO-DMT or a pharmaceutically acceptable salt thereof.

44. 5-Methoxy-N,N-dimethyltryptamine (5-MeO-DMT) or a pharmaceutically acceptable salt thereof for use in any of claims 1 to 40, wherein the patient is a breastfeeding mother who is advised to discontinue breastfeeding until 3 hours after the last administration of 5-MeO-DMT or a pharmaceutically acceptable salt thereof.

45. 5-Methoxy-N,N-dimethyltryptamine (5-MeO-DMT) or a pharmaceutically acceptable salt thereof for use in any of claims 1 to 40, wherein the patient is a breastfeeding mother who is advised to discontinue breastfeeding until 2 hours after the last administration of 5-MeO-DMT or a pharmaceutically acceptable salt thereof.

46. 5-MeO-DMT or a pharmaceutically acceptable salt thereof for use as in claims 1 to 45, wherein the treatment improves maternal functioning.

47. 5-MeO-DMT or a pharmaceutically acceptable salt thereof for use as in claim 46, wherein the improvement relates to one or more, in particular two or more functional domains according to the Barkin Index of Maternal Functioning (BIMF) selected from self-care, infant care, mother-child interaction, psychological wellbeing of the mother, social support, management, and adjustment.

48. 5-MeO-DMT or a pharmaceutically acceptable salt thereof for use as in claim 46 or 47, wherein the BIMF score is improved by 10% or more, preferably by 20% or more.
